(19) United States
(12) Patent Application Publication
(10) Pub. No.: US 2023/0051811 A1
(43) Pub. Date: Feb. 16, 2023

(54) RECTAL DELIVERY OF MESSENGER RNA

(71) Applicant: Translate Bio, Inc, Waltham, MA (US)

(72) Inventors: Shrirang Karve, Waltham, MA (US); Frank DeRosa, Waltham, MA (US); Ashish Sarode, Waltham, MA (US)

(21) Appl. No.: 17/786,971

(22) PCT Filed: Dec. 18, 2020
(86) PCT No.: PCT/US2020/065945

§ 371 Date: Jun. 17, 2022

Related U.S. Application Data
(60) Provisional application No. 62/951,844, filed on Dec. 20, 2019.

(51) Int. Cl. A61K 9/00; A61K 48/00; A61K 9/127; A61K 9/02; A61K 9/51

(52) U.S. Cl. CPC A61K 9/0031; A61K 48/0041; A61K 9/1271; A61K 9/02; A61K 9/5123; A61K 9/5146

(57) ABSTRACT

The present invention provides, among other things, effective methods and compositions for delivering messenger RNA (mRNA) via rectal delivery. The present invention is, in part, based on unexpected observation that mRNA may be effectively delivered to the circulation, liver, kidney, colon and/or rectum via rectal delivery despite the barriers such as RNase and mucus layer.

### CROSS-REFERENCE TO RELATED APPLICATIONS

This application claims the benefit of, and priority to, U.S. Ser. No. 62/951,844 filed on Dec. 20, 2019, the contents of which are incorporated herein.

### BACKGROUND

Delivery of nucleic acids, especially messenger RNA (mRNA), to target cells and tissues remains a technical challenge. Various difficulties are encountered with delivery of mRNA to cells of interest, including for example, physical and chemical barriers. These difficulties are encountered using a wide-variety of delivery methods such as parenteral and oral routes of delivery. Rectal delivery is particularly challenging at least in part due to the unique composition of the rectum and colon, such as the presence of RNase in the rectum.

### SUMMARY OF INVENTION

The present invention provides, among other things, effective methods and compositions for delivering messenger RNA (mRNA) via rectal delivery. The present invention is, in part, based on the surprising discovery that lipid encapsulated mRNA can be effectively delivered to the circulation, liver, kidney, intestine, colon and/or rectum via mucosal delivery, including rectal delivery, despite numerous barriers such as RNase and mucosal layers.

In some aspects, the invention provides a method for delivery of messenger RNA (mRNA) to a subject for in vivo production of a protein or a peptide in the subject, comprising administering to the subject by rectal delivery, a composition comprising an mRNA that encodes a protein or a peptide and is encapsulated within a lipid nanoparticle and wherein the administering of the composition results in expression of the protein or the peptide encoded by the mRNA that is detectable in the subject at least about 24 hours, about 48 hours, about 72 hours, or about 96 hours after administration.

In some embodiments, the protein or the peptide encoded by the mRNA is detectable in the subject's circulation at least about 24 hours, about 48 hours, about 72 hours, or about 96 hours after administration. Accordingly, in some embodiments, the protein or the peptide encoded by the mRNA is detectable in the subject's circulation at least about 24 hours after administration. In some embodiments, the protein or the peptide encoded by the mRNA is detectable in the subject's circulation at least about 48 hours after administration. In some embodiments, the protein or the peptide encoded by the mRNA is detectable in the subject's circulation at least about 72 hours after administration. In some embodiments, the protein or the peptide encoded by the mRNA is detectable in the subject's circulation at least about 96 hours after administration.

In some embodiments, the protein or the peptide encoded by the mRNA is detectable in the subject's liver at least about 24 hours, about 48 hours, about 72 hours, or about 96 hours after administration. Accordingly, in some embodiments, the protein or peptide encoded by the mRNA is detectable in the subject's liver at least about 24 hours after administration. In some embodiments, the protein or peptide encoded by the mRNA is detectable in the subject's liver at least about 48 hours after administration. In some embodiments, the protein or peptide encoded by the mRNA is detectable in the subject's liver at least about 72 hours after administration. in some embodiments, the protein or peptide encoded by the mRNA is detectable in the subject's liver at least about 96 hours after administration.

In some embodiments, the protein or the peptide encoded by the mRNA is detectable in the subject's kidney at least about 24 hours, about 48 hours, about 72 hours, or about 96 hours after administration. Accordingly, in some embodiments, the protein or the peptide encoded by the mRNA is detectable in the subject's kidney at least about 24 hours after administration. In some embodiments, the protein or the peptide encoded by the mRNA is detectable in the subject's kidney at least about 48 hours after administration. In some embodiments, the protein or the peptide encoded by the mRNA is detectable in the subject's kidney at least about 72 hours after administration. In some embodiments, the protein or the peptide encoded by the mRNA is detectable in the subject's kidney at least about 96 hours after administration.

In some embodiments, the protein or the peptide encoded by the mRNA is detectable in the subject's colon at least about 24 hours, about 48 hours, about 72 hours, or about 96 hours after administration. Accordingly, in some embodiments, the protein or the peptide encoded by the mRNA is detectable in the subject's colon at least about 24 hours after administration. In some embodiments, the protein or the peptide encoded by the mRNA is detectable in the subject's colon at least about 48 hours after administration. In some embodiments, the protein or the peptide encoded by the mRNA is detectable in the subject's colon at least about 72 hours after administration. In some embodiments, the protein or the peptide encoded by the mRNA is detectable in the subject's colon at least about 96 hours after administration.

In some embodiments, the protein or the peptide encoded by the mRNA is detectable in the subject's rectum at least about 24 hours, about 48 hours, about 72 hours, or about 96 hours after administration. Accordingly, in some embodiments, the protein or the peptide encoded by the mRNA is detectable in the subject's rectum at least about 24 hours after administration. In some embodiments, the protein or the peptide encoded by the mRNA is detectable in the subject's rectum at least about 48 hours after administration. In some embodiments, the protein or the peptide encoded by the mRNA is detectable in the subject's rectum at least about 72 hours after administration. In some embodiments, the protein or the peptide encoded by the mRNA is detectable in the subject's rectum at least about 96 hours after administration. I

In some embodiments, the in vivo production of the protein or the peptide is in the subject's circulation, liver, kidney, colon and/or rectum. Accordingly, in some embodiments, the in vivo production of the protein or the peptide is in the subject's circulation. In some embodiments, the in vivo production of the protein or the peptide is in the subject's liver. In some embodiments, the in vivo production of the protein or the peptide is in the subject's kidney. In some embodiments, the in vivo production of the protein or the peptide is in the subject's colon. In some embodiments, the in vivo production of the protein or the peptide is in the subject's rectum.

In some embodiments, the lipid nanoparticle comprises one or more cationic lipids, one or more non-cationic lipids and one or more PEG-modified lipids. Accordingly, in some embodiments, the lipid nanoparticle comprises one or more cationic lipids. In some embodiments, the lipid nanoparticle comprises one or more non-cationic lipids. In some embodiments, the lipid nanoparticle comprises one or more PEG-modified lipids. In some embodiments, the lipid nanoparticle comprises one

In some embodiments, the lipid nanoparticle comprises cholesterol.

In some embodiments, the rectal delivery is by suppository, enema, catheter or a bulb syringe. Accordingly, in some embodiments the rectal delivery is by suppository. In some embodiments, the rectal delivery is by enema. In some embodiments, the rectal delivery is by catheter. In some embodiments, the rectal delivery is by a bulb syringe.

In some embodiments, the rectal delivery is by suppository.

In some embodiments, the composition does not comprise a lipid-based suppository component.

In some embodiments, the lipid-based suppository component is cocoa butter, theobroma oil, synthetic fats or synthetic bases. Accordingly, in some embodiments, the lipid-based suppository component is cocoa butter. In some embodiments, the lipid-based suppository component is theobroma oil. In some embodiments, the lipid-based suppository component is a synthetic fat. In some embodiments, the lipid-based suppository component is a synthetic base.

In some embodiments, the composition comprises a permeability enhancer.

In some embodiments, the permeability enhancer is selected from bile salts, surfactants, fatty acids and derivatives, glycerides, chelators, salicylates, or polymers. Accordingly, in some embodiments, the permeability enhancer is a bile salt. In some embodiments, the permeability enhancer is a fatty acid and derivatives. In some embodiments, the permeability enhancer is a glyceride. In some embodiments, the permeability enhancer is a chelator. In some embodiments, the permeability enhancer is a salicylate. In some embodiments, the permeability enhancer is a polymer.

In some embodiments, the fatty acids and derivatives are selected from sorbitan laurate, sodium caprate, sucrose, palitate, lauroyl choline, sodium myristate, or palmitoyl carnitine. Accordingly, in some embodiments, the fatty acid and derivatives is sorbitan laurate. In some embodiments, the fatty acid and derivatives include sodium caprate. In some embodiments, the fatty acid and derivatives is sucrose. In some embodiments, the fatty acid and derivatives is palitate. In some embodiments, the fatty acid and derivatives is lauroyl choline. In some embodiments, the fatty acid and derivatives is sodium myristate. In some embodiments, the fatty acid and derivatives is palmitoyl carnitine

In some embodiments, the permeability enhancer is a form of caprate.

In some embodiments, the caprate-based permeability enhancer is sodium caprate.

In some embodiments, the permeability enhancer is Labrasol®.

In some embodiments, the composition comprises a water-based suppository component.

In some embodiments, the water-based suppository component is selected from glycerin, gelatin or polyethylene glycol (PEG), or combinations thereof. In some embodiments, the water-based suppository component is glycerin. In some embodiments, the water-based suppository component is gelatin. In some embodiments, the water-based suppository component is polyethylene glycol (PEG).

In some embodiments, the composition further comprises gelatin.

In some embodiments, the only water-based suppository component is gelatin.

In some embodiments, the composition comprises about 5% or more gelatin in water, 10% or more gelatin in water, 20% or more gelatin in water, 30% or more gelatin in water, or 50% or more gelatin in water. Accordingly, in some embodiments, the composition comprises about 5% or more gelatin in water. For example, in some embodiments, the composition comprises about 5%, 6%, 7%, 8% or 9% or more gelatin. In some embodiments, the composition comprises about 10% or more gelatin in water. For example, in some embodiments, the composition comprises about 10%, 11%, 12%, 13%, 14%, 15%, 16%, 17%, 18% or 19% or more gelatin in water. In some embodiments, the composition comprises about 20% or more gelatin in water. For example, in some embodiments, the composition comprises about 20%, 21%, 22%, 23%, 24%, 25%, 26%, 27%, 28%, or 29% or more gelatin in water. In some embodiments, the composition comprises about 30% or more gelatin in water. For example, in some embodiments, the composition comprises about 30%, 31%, 32%, 33%, 34%, 35%, 36%, 37%, 38%, 39%, 40%, 41%, 42%, 43%, 44%, 45%, 46%, 47%, 48%, or 49% or more gelatin in water. In some embodiments, the composition comprises about 50% or more gelatin in water. For example, in some embodiments, the composition comprises about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more gelatin in water.

In some embodiments, the composition further comprises 0.25 mg/mL or greater mRNA, 0.5 mg/mL or greater mRNA, 0.75 mg/mL or greater mRNA, or 1 mg/mL or greater mRNA. Accordingly, in some embodiments, the composition further comprises 0.25 mg/mL or greater mRNA. In some embodiments, the composition comprises 0.5 mg/mL or greater mRNA. In some embodiments, the composition comprises 0.75 mg/mL or greater mRNA. In some embodiments, the composition comprises 1 mg/mL or greater mRNA.

In some embodiments, the composition comprises 0.5 mg or greater mRNA, 0.75 mg or greater mRNA, 1 mg or greater mRNA, 1.25 mg or greater mRNA, 1.5 mg or greater mRNA, or 1.75 mg or greater mRNA. Accordingly, in some embodiments, the composition comprises 0.5 mg or greater mRNA. In some embodiments, the composition comprises 0.75 mg or greater mRNA. In some embodiments, the composition comprises 1 mg or greater mRNA. In some embodiments, the composition comprises 1.25 mg or greater mRNA. In some embodiments, the composition comprises 1.5 mg or greater mRNA. In some embodiments, the composition comprises 1.75 mg or greater mRNA.

In some embodiments, the composition is formulated for a suppository of about 3 grams, about 2 grams, or about 1 gram. Accordingly, in some embodiments, the composition is formulated for a suppository of about 3 grams. In some embodiments, the composition is formulated for a suppository of about 2 grams. In some embodiments, the composition is formulated for a suppository of about 1 gram.

In some embodiments, the composition is formulated for a suppository having a volume of about 2.0 mL, about 3.5 mL, about 7.5 mL, or about 10.0 mL. Accordingly, in some embodiments, the composition is formulated for a suppository having a volume of about 2.0 mL. In some embodiments, the composition is formulated for a suppository having a volume of about 3.5 mL. In some embodiments, the composition is formulated for a suppository having a volume of about 7.5 mL. In some embodiments, the composition is formulated for a suppository having a volume of about 10.0 mL.

In some embodiments, the suppository is refrigerated prior to administration.

In some embodiments, the subject is first administered a permeability enhancer prior to the administering of the composition comprising mRNA.

In some embodiments, the permeability enhancer is administered to the subject about 30 minutes, about 1 hour, about 2.5 hours, about 5 hours, or about 12 hours prior to administering the composition comprising mRNA. Accordingly, in some embodiments, the permeability enhancer is administered to the subject about 30 minutes prior to administering the composition comprising mRNA. In some embodiments, the permeability enhancer is administered to the subject about 1 hour prior to administering the composition comprising mRNA. In some embodiments, the permeability enhancer is administered to the subject about 2.5 hours prior to administering the composition comprising mRNA. In some embodiments, the permeability enhancer is administered to the subject about 5.0 hours prior to administering the composition comprising mRNA. In some embodiments, the permeability enhancer is administered to the subject about 12 hours prior to administering the composition comprising mRNA.

In some aspects, the invention provides a method of delivery of messenger RNA (mRNA) to a subject for in vivo production of a protein or peptide in the subject, comprising administering to the subject by mucosal delivery a composition comprising an mRNA that encodes a protein or a peptide and is encapsulated within a lipid nanoparticle, and wherein the administering of the composition results in expression of the protein or peptide encoded by the mRNA that is detectable in the subject at least about 24 hours, about 48 hours, about 72 hours, or about 96 hours after administration. Accordingly, in some embodiments, the administering of the composition results in expression of the protein or peptide encoded by the mRNA that is detectable in the subject at least about 24 hour after administration. In some embodiments, the administering of the composition results in expression of the protein or peptide encoded by the mRNA that is detectable in the subject at least about 48 hour after administration. In some embodiments, the administering of the composition results in expression of the protein or peptide encoded by the mRNA that is detectable in the subject at least about 72 hour after administration. In some embodiments, the administering of the composition results in expression of the protein or peptide encoded by the mRNA that is detectable in the subject at least about 96 hour after administration.

In some embodiments, the mRNA is detectable in the subject's circulation, liver, kidney, colon, and/or rectum at least about 24 hours, about 48 hours, about 72 hours, or about 96 hours after administration. Accordingly, in some embodiments the mRNA is detectable in the subject's circulation at least about 24 hours after administration. In some embodiments, the mRNA is detectable in the subject's circulation at least about 48 hours after administration. In some embodiments, the mRNA is detectable in the subject's circulation at least about 72 hours after administration. In some embodiments, the mRNA is detectable in the subject's circulation at least about 96 hours after administration. In some embodiments, the mRNA is detectable in the subject's liver at least about 24 hours after administration. In some embodiments, the mRNA is detectable in the subject's liver at least about 48 hours after administration. In some embodiments, the mRNA is detectable in the subject's liver at least about 72 hours after administration. In some embodiments, the mRNA is detectable in the subject's liver at least about 96 hours after administration. In some embodiments, the mRNA is detectable in the subject's kidney at least about 24 hours after administration. In some embodiments, the mRNA is detectable in the subject's kidney at least about 48 hours after administration. In some embodiments, the mRNA is detectable in the subject's kidney at least about 72 hours after administration. In some embodiments, the mRNA is detectable in the subject's kidney at least about 96 hours after administration. In some embodiments, the mRNA is detectable in the subject's colon at least about 24 hours after administration. In some embodiments, the mRNA is detectable in the subject's colon at least about 48 hours after administration. In some embodiments, the mRNA is detectable in the subject's colon at least about 72 hours after administration. In some embodiments, the mRNA is detectable in the subject's colon at least about 96 hours after administration. In some embodiments, the mRNA is detectable in the subject's rectum at least about 24 hours after administration. In some embodiments, the mRNA is detectable in the subject's rectum at least about 48 hours after administration. In some embodiments, the mRNA is detectable in the subject's rectum at least about 72 hours after administration. In some embodiments, the mRNA is detectable in the subject's rectum at least about 96 hours after administration.

In some embodiments, the mucosal delivery is rectal, vaginal, ocular, oral, or gastrointestinal. Accordingly, in some embodiments, the mucosal delivery is rectal. In some embodiments, the mucosal delivery is vaginal. In some embodiments, the mucosal delivery is ocular. In some embodiments, the mucosal delivery is oral. In some embodiments, the mucosal delivery is gastrointestinal.

In some embodiments, the oral delivery is buccal or sublingual. Accordingly, in some embodiments, the oral delivery is buccal. In some embodiments, the oral delivery is sublingual.

In some embodiments, the in vivo production of the protein or peptide is in the subject's circulation, liver, kidney, colon and/or rectum. Accordingly, in some embodiments, the in vivo production of the protein or peptide is in the subject's circulation. In some embodiments, the in vivo production of the protein or peptide is in the subject's liver. In some embodiments, the in vivo production of the protein or peptide is in the subject's kidney. In some embodiments, the in vivo production of the protein or peptide is in the subject's colon. In some embodiments, the in vivo production of the protein or peptide is in the subject's rectum.

In some aspects, the invention provides a suppository for rectal administration of mRNA, the suppository comprising: mRNA encapsulated within a lipid nanoparticle, wherein the mRNA encodes a protein or peptide; and gelatin.

In some embodiments, the suppository comprises about 5% or more gelatin in water, 10% or more gelatin in water, 20% or more gelatin in water, 30% or more gelatin in water, or 50% or more gelatin in water. Accordingly, in some embodiments, the suppository comprises about 5% or more gelatin in water. For example, in some embodiments, the suppository comprises about 5%, 6%, 7%, 8% or 9% or more gelatin. In some embodiments, the suppository comprises about 10% or more gelatin in water. For example, in some embodiments, the suppository comprises about 10%, 11%, 12%, 13%, 14%, 15%, 16%, 17%, 18% or 19% or more gelatin in water. In some embodiments, the suppository comprises about 20% or more gelatin in water. For example, in some embodiments, the suppository comprises about 20%, 21%, 22%, 23%, 24%, 25%, 26%, 27%, 28%, or 29% or more gelatin in water. In some embodiments, the suppository comprises about 30% or more gelatin in water. For example, in some embodiments, the suppository comprises about 30%, 31%, 32%, 33%, 34%, 35%, 36%, 37%, 38%, 39%, 40%, 41%, 42%, 43%, 44%, 45%, 46%, 47%, 48%, or 49% or more gelatin in water. In some embodiments, the suppository comprises about 50% or more gelatin in water. For example, in some embodiments, the suppository comprises about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more gelatin in water.

In some embodiments, the suppository does not comprise a lipid-based suppository component.

In some embodiments, the lipid-based suppository component is cocoa butter, theobroma oil, synthetic fats or synthetic bases. Accordingly, in some embodiments, the lipid-based suppository component is cocoa butter. In some embodiments, the lipid-based suppository component is theobroma oil. In some embodiments, the lipid-based suppository component is a synthetic fat. In some embodiments, the lipid-based suppository component is a synthetic base. In some embodiments, the lipid-based suppository component is cocoa butter, theobroma oil, synthetic fats or synthetic bases, or any combination thereof.

In some embodiments, the suppository comprises a permeability enhancer.

In some embodiments, the suppository comprises a permeability enhancer selected from bile salts, surfactants, fatty acids and derivatives, glycerides, chelators, salicylates, or polymers. Accordingly, in some embodiments, the permeability enhancer is a bile salt. In some embodiments, the permeability enhancer is a fatty acid and derivatives. In some embodiments, the permeability enhancer is a glyceride. In some embodiments, the permeability enhancer is a chelator. In some embodiments, the permeability enhancer is a salicylate. In some embodiments, the permeability enhancer is a polymer.

In some embodiments, the suppository comprises fatty acids and derivatives that are selected from sorbitan laurate, sodium caprate, sucrose, palitate, lauroyl choline, sodium myristate, or palmitoyl carnitine. Accordingly, in some embodiments, the fatty acid and derivatives include sodium caprate. In some embodiments, the fatty acid and derivatives is sucrose. In some embodiments, the fatty acid and derivatives is palitate. In some embodiments, the fatty acid and derivatives is lauroyl choline. In some embodiments, the fatty acid and derivatives is sodium myristate. In some embodiments, the fatty acid and derivatives is palmitoyl carnitine

In some embodiments, the suppository comprises a permeability enhancer that is a form of caprate.

In some embodiments, the caprate-based permeability enhancer is sodium caprate.

In some embodiments, the permeability enhancer is Labrasol®.

In some embodiments, the suppository further comprises glycerin and/or PEG. Accordingly, in some embodiments, the suppository further comprises glycerin. In some embodiments, the suppository further comprise PEG.

In some embodiments, the suppository softens or melts at about between 36 and 37° C. Accordingly, in some embodiments, the suppository softens at about 36.0° C., 36.1° C., 36.2° C., 36.3° C., 36.4° C., 36.5° C., 36.6° C., 36.7° C., 36.8° C., 36.9° C., or 37.0° C.

In some embodiments, the protein or the peptide encoded by the mRNA is detectable in the subject's liver at least about 24 hours, about 48 hours, about 72 hours, or about 96 hours after administration.

### BRIEF DESCRIPTION OF THE DRAWINGS

The following figures are for illustration purposes only and not for limitation.

FIG. 1 depicts an exemplary suppository comprising mRNA encapsulated within lipid nanoparticles. The suppository can be delivered rectally.

FIG. 2A depicts an exemplary imaging of mice after 24 hours of the rectal administration of saline as a negative control. FIG. 2B depicts an exemplary imaging of various tissues after 24 hours of the rectal administration of saline as a negative control. No signal was detected when saline was administered rectally.

FIG. 3A depicts an exemplary imaging of mice after 24 hours of the rectal administration of FFLuc mRNA-LNP. FIG. 3B depicts an exemplary imaging of various tissues after 24 hours of the rectal administration of FFLuc mRNA-LNP. Signal of luciferase activity was detected in mice. Colon showed strong luminescence.

FIG. 4 depicts an exemplary imaging of mice after 24 hours of the rectal administration of FFLuc mRNA-LNP at 0.2 mg dose (Group 1) or at 0.05 mg dose (Group 2). Mice in Group 2 were pre-dosed with sodium caprate prior to the administration of mRNA-LNP.

FIG. 5 is an exemplary graphical representation of luminescence detected for mice at 24 hours post administration of saline (negative control), 0.2 mg dose of mRNA-LNP (Group 1), or 0.05 mg dose of mRNA-LNP with sodium caprate (Group 2).

FIG. 6A depicts an exemplary imaging of mice at 24 hours post rectal administration of suppository comprising FFLuc mRNA-LNP. FIG. 6B depicts an exemplary imaging of various tissues of rats after 24 hours of the rectal administration of the suppository.

FIG. 7 is an exemplary graphical representation of hEPO protein in serum detected in rats at x hours post administration of the composition.

### DEFINITIONS

In order for the present invention to be more readily understood, certain terms are first defined below. Additional definitions for the following terms and other terms are set forth throughout the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Comprising: As used herein, the term “comprising,” or variations such as “comprises” or “comprising,” will be understood to imply the inclusion of a stated element, integer or step, or group of elements, integers or steps, but not the exclusion of any other element, integer or step, or group of elements, integers or steps.

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”). Other exemplary situations include one in which an mRNA is delivered to a target tissue and the encoded protein is expressed and secreted into patient's circulation system (e.g., serum) and systematically distributed and taken up by other tissues (also referred to as “systemic distribution” or “systemic delivery). In other exemplary situations, the mRNA is delivered systemically and is taken up in a wide variety of cells and tissues in vivo. In some exemplary situations, the delivery is intravenous, intramuscular or subcutaneous.

Dosing interval: As used herein dosing interval in the context of a method for treating a disease is the frequency of administering a therapeutic composition in a subject (mammal) in need thereof, for example an mRNA composition, at an effective dose of the mRNA, such that one or more symptoms associated with the disease is reduced; or one or more biomarkers associated with the disease is reduced, at least for the period of the dosing interval. Dosing frequency and dosing interval may be used interchangeably in the current disclosure.

Efficacy: As used herein, the term “efficacy,” or grammatical equivalents, refers to an improvement of a biologically relevant endpoint, as related to delivery of mRNA that encodes a relevant protein or peptide. In some embodiments, the biological endpoint is protecting against an ammonium chloride challenge at certain time points after administration.

Encapsulation: As used herein, the term “encapsulation,” or its grammatical equivalent, refers to the process of confining a nucleic acid molecule within a nanoparticle.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and their grammatical equivalents, are used interchangeably.

Effective dose: As used herein, an effective dose is a dose of the mRNA in the pharmaceutical composition which when administered to the subject in need thereof, hereby a mammalian subject, according to the methods of the invention, is effective to bring about an expected outcome in the subject, for example reduce a symptom associated with the disease.

Functional: As used herein, a “functional” biological molecule is a biological molecule in a form in which it exhibits a property and/or activity by which it is characterized.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Liposome: As used herein, the term “liposome” refers to any lamellar, multilamellar, or solid nanoparticle vesicle. Typically, a liposome as used herein can be formed by mixing one or more lipids or by mixing one or more lipids and polymer(s). In some embodiments, a liposome suitable for the present invention contains a cationic lipids(s) and optionally non-cationic lipid(s), optionally cholesterol-based lipid(s), and/or optionally PEG-modified lipid(s).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

N/P Ratio: As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In specific embodiments, a patient is a human. A human includes pre- and post-natal forms.

Polypeptide: As used herein, a “polypeptide”, generally speaking, is a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides sometimes include “non-natural” amino acids or other entities that nonetheless are capable of integrating into a polypeptide chain, optionally.

Protein: As used herein, the term “protein” of “therapeutic protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Target tissues: As used herein, the term “target tissues” refers to any tissue that is affected by a disease to be treated. In some embodiments, target tissues include those tissues that display disease-associated pathology, symptom, or feature.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Unless otherwise defined, all technical and scientific terms used herein have the same meaning as commonly understood by one of ordinary skill in the art to which this application belongs and as commonly used in the art to which this application belongs; such art is incorporated by reference in its entirety. In the case of conflict, the present Specification, including definitions, will control.

### DETAILED DESCRIPTION

The present invention provides, among other things, effective methods and compositions for delivering messenger RNA (mRNA) and/or its protein or polypeptide product to a subject via a mucosal route, for example through rectal delivery. The present invention is, in part, based on a surprising finding that mRNA and/or its protein or polypeptide product may be effectively delivered to the subject's circulation, liver, kidney, colon and/or rectum via rectal delivery despite numerous chemical and physical barriers.

Various aspects of the invention are described in detail in the following sections. The use of sections is not meant to limit the invention. Each section can apply to any aspect of the invention. In this application, the use of “or” means “and/or” unless stated otherwise.

Mucosal Delivery of Lipid-Encapsulated mRNA

The invention provides various methods of mRNA delivery to target tissue. The delivery methods include administration of lipid-encapsulated mRNA across any mucosal tissue. For example, the lipid-encapsulated mRNA is delivered via a rectal, vaginal, ocular, oral, and/or gastrointestinal route.

In some aspects, the invention provides among other things, a method of delivery of messenger RNA (mRNA) to a subject for in vivo production of a protein or a peptide in the subject, comprising administering to the subject by mucosal delivery a composition comprising an mRNA that encodes a protein or a peptide and is encapsulated within a lipid nanoparticle, and wherein the administering of the composition results in expression of the protein or the peptide encoded by the mRNA that is detectable in the subject at least about 24 hours, about 48 hours, about 72 hours, or about 96 hours after administration.

In some embodiments, the mucosal delivery of lipid-encapsulated mRNA is via the rectum.

Rectal Delivery

In some embodiments, the invention provides a method for rectal delivery of lipid-encapsulated mRNA that encodes a protein or a peptide of interest.

The advantages of rectal delivery include the ease of administration, allowing the patients to remain in a home setting. The inpatient environment and special formulation of sterile medications required by the intravenous administration are not necessary for rectal delivery. A therapeutic composition can be administered rectally via a suppository, an enema, a bulb syringe, and a catheter. Rectal administration using a specialized rectal catheter can be placed by a clinician in the home. Many oral forms of medications can be crushed and suspended in water to be given via a rectal catheter. As such, rectal administration is especially safe and convenient for infants and elderly, and useful for patients with aversion to needles or with any digestive tract motility problem, such as dysphagia, ileus, or bowel obstruction that would interfere with the progression of the medication through the tract. Furthermore, rectally administered drugs generally have faster onset and higher bioavailability, and are less prone to nausea compared to oral drug administration. Rectally administered drugs bypass about two thirds of first-pass metabolism, resulting in less alteration and greater concentration of the drug in the patient's circulatory system. However, delivery mRNA via rectal route is extremely challenging. The rectal area (i.e., rectum and colon) has high amounts of RNase which would degrade mRNA instantly. Furthermore, the mucus layer in the rectum and/or colon would act as an absorption barrier. Moreover, fecal impaction can impede rectal delivery of a drug.

Despite these challenges, the methods provided herein allow for delivering messenger RNA (mRNA) via a rectal route. The present invention is, in part, based on surprising discovery that lipid encapsulated mRNA can be effectively delivered to a subject's the circulation, liver, kidney, colon and/or rectum via rectal delivery despite numerous barriers such as the presence of RNase, a mucus layer, and fecal impaction. Such non-invasive routes of delivery unexpectedly provide an effective means to conveniently deliver lipid-encapsulated therapeutic compositions.

The present invention provides, among other things, a method for delivery of messenger RNA (mRNA) to a subject for in vivo production of a protein or a peptide in the subject, comprising administering to the subject by rectal delivery, a composition comprising an mRNA that encodes a protein or a peptide and is encapsulated within a lipid nanoparticle and wherein the administering of the composition results in expression of the protein or the peptide encoded by the mRNA that is detectable in the subject at least about 24 hours, about 48 hours, about 72 hours, or about 96 hours after administration. This method allows for the delivery of lipid encapsulated mRNA via a rectal route that results in expression of the protein or the peptide encoded by the mRNA in various tissues in the recipient subject. For example, the method allows for expression of the protein or the peptide encoded by the mRNA in the subject's liver, kidney, circulation, colon or rectum.

The methods described herein are suitable for administration of the lipid-encapsulated mRNA in the home setting. In some embodiments, the rectal delivery is by suppository, enema, catheter or a bulb syringe. In some embodiments, the rectal delivery is by suppository. In some embodiments, the rectal delivery is by enema. In some embodiments, the rectal delivery is by catheter. Various kinds of specialized catheters can be used with the methods disclosed herein, for example, one such specialized catheter is the Macy Catheter. In some embodiments, the rectal delivery is by a bulb syringe.

In some embodiments, the compositions of the invention are delivered to various target tissues in the subject. Thus, the present invention can be used as a non-invasive means of facilitating delivery of a desired protein or peptide, and/or the production of proteins or peptides encoded thereby at a target tissue. The methods and composition described herein are useful in the management and treatment of a large number of diseases, which result from both secreted and non-secreted protein and/or enzyme deficiencies.

In some embodiments, rectal delivery of lipid-encapsulated mRNA results in the production of a desired protein or peptide encoded by the mRNA in the circulation, liver, kidney, colon, rectum, heart and/or spleen. Accordingly, in some embodiments, the lipid-encapsulated mRNA results in the production of a desired protein or peptide encoded by the mRNA in the circulation. In some embodiments, the lipid-encapsulated mRNA results in the production of a desired protein or peptide encoded by the mRNA in the liver. In some embodiments, the lipid-encapsulated mRNA results in the production of a desired protein or peptide encoded by the mRNA in the kidney. In some embodiments, the lipid-encapsulated mRNA results in the production of a desired protein or peptide encoded by the mRNA in the colon. In some embodiments, the lipid-encapsulated mRNA results in the production of a desired protein or peptide encoded by the mRNA in the rectum. In some embodiments, the lipid-encapsulated mRNA results in the production of a desired protein or peptide encoded by the mRNA in the heart. In some embodiments, the lipid-encapsulated mRNA results in the production of a desired protein or peptide encoded by the mRNA in the spleen.

In some embodiments, rectal delivery of lipid-encapsulated mRNA results in the production of a desired protein or peptide encoded by the mRNA in the circulation, liver, kidney, colon, rectum, heart, and/or spleen. In some embodiments, the protein or the peptide encoded by the mRNA is produced in the subject at least about 6 hours, about 12 hours, about 18 hours, about 24 hours, about 36 hours, about 48 hours, about 60 hours, about 72 hours, about 96 hours, about 120 hours, about 144 hours or about 168 hours after administration. In some embodiments, the protein or the peptide encoded by the mRNA is produced in the subject at least about 6 hours after administration. In some embodiments, the protein or the peptide encoded by the mRNA is produced in the subject at least about 12 hours after administration. In some embodiments, the protein or the peptide encoded by the mRNA is produced in the subject at least about 18 hours after administration. In some embodiments, the protein or the peptide encoded by the mRNA is produced in the subject at least about 24 hours after administration. In some embodiments, the protein or the peptide encoded by the mRNA is produced in the subject at least about 36 hours after administration. In some embodiments, the protein or the peptide encoded by the mRNA is produced in the subject at least about 48 hours after administration. In some embodiments, the protein or the peptide encoded by the mRNA is produced in the subject at least about 60 hours after administration. In some embodiments, the protein or the peptide encoded by the mRNA is produced in the subject at least about 72 hours after administration. In some embodiments, the protein or the peptide encoded by the mRNA is produced in the subject at least about 96 hours after administration. In some embodiments, the protein or the peptide encoded by the mRNA is produced in the subject at least about 120 hours after administration. In some embodiments, the protein or the peptide encoded by the mRNA is produced in the subject at least about 144 hours after administration. In some embodiments, the protein or the peptide encoded by the mRNA is produced in the subject at least about 168 hours after administration.

In some embodiments, the protein or the peptide encoded by the mRNA is produced in the subject at least about 1 day, 2 days, 3 days, 4 days, 5, days, 6 days, 7 days, 8 days, 9 days or 10 days after administration. In some embodiments, the protein or the peptide encoded by the mRNA is produced in the subject at least about 1 day after administration. In some embodiments, the protein or the peptide encoded by the mRNA is produced in the subject at least about 2 days after administration. In some embodiments, the protein or the peptide encoded by the mRNA is produced in the subject at least about 3 days after administration. In some embodiments, the protein or the peptide encoded by the mRNA is produced in the subject at least about 4 days after administration. In some embodiments, the protein or the peptide encoded by the mRNA is produced in the subject at least about 5 days after administration. In some embodiments, the protein or the peptide encoded by the mRNA is produced in the subject at least about 6 days after administration. In some embodiments, the protein or the peptide encoded by the mRNA is produced in the subject at least about 7 days after administration. In some embodiments, the protein or the peptide encoded by the mRNA is produced in the subject at least about 8 days after administration. In some embodiments, the protein or the peptide encoded by the mRNA is produced in the subject at least about 9 days after administration. In some embodiments, the protein or the peptide encoded by the mRNA is produced in the subject at least about 10 days after administration.

In some embodiments, rectal delivery of lipid-encapsulated mRNA results in the detection of a desired protein or peptide encoded by the mRNA in the circulation, liver, kidney, colon, rectum, heart and/or spleen. Accordingly, in some embodiments, rectal delivery of lipid-encapsulated mRNA results in the detection of a desired protein or peptide encoded by the mRNA in the circulation. In some embodiments, rectal delivery of lipid-encapsulated mRNA results in the detection of a desired protein or peptide encoded by the mRNA in the liver. In some embodiments, rectal delivery of lipid-encapsulated mRNA results in the detection of a desired protein or peptide encoded by the mRNA in the kidney. In some embodiments, rectal delivery of lipid-encapsulated mRNA results in the detection of a desired protein or peptide encoded by the mRNA in the colon. In some embodiments, rectal delivery of lipid-encapsulated mRNA results in the detection of a desired protein or peptide encoded by the mRNA in the rectum. In some embodiments, rectal delivery of lipid-encapsulated mRNA results in the detection of a desired protein or peptide encoded by the mRNA in the heart. In some embodiments, rectal delivery of lipid-encapsulated mRNA results in the detection of a desired protein or peptide encoded by the mRNA in the spleen.

In some embodiments, the protein or the peptide encoded by the mRNA is detectable in the subject at least about 6 hours, about 12 hours, about 18 hours, about 24 hours, about 36 hours, about 48 hours, about 60 hours, about 72 hours, about 96 hours, about 120 hours, about 144 hours or about 168 hours after administration. In some embodiments, the protein or the peptide encoded by the mRNA is detectable in the subject at least about 6 hours after administration. In some embodiments, the protein or the peptide encoded by the mRNA is detectable in the subject at least about 12 hours after administration. In some embodiments, the protein or the peptide encoded by the mRNA is detectable in the subject at least about 18 hours after administration. In some embodiments, the protein or the peptide encoded by the mRNA is detectable in the subject at least about 24 hours after administration. In some embodiments, the protein or the peptide encoded by the mRNA is detectable in the subject at least about 36 hours after administration. In some embodiments, the protein or the peptide encoded by the mRNA is detectable in the subject at least about 48 hours after administration. In some embodiments, the protein or the peptide encoded by the mRNA is detectable in the subject at least about 60 hours after administration. In some embodiments, the protein or the peptide encoded by the mRNA is detectable in the subject at least about 72 hours after administration. In some embodiments, the protein or the peptide encoded by the mRNA is detectable in the subject at least about 96 hours after administration. In some embodiments, the protein or the peptide encoded by the mRNA is detectable in the subject at least about 120 hours after administration. In some embodiments, the protein or the peptide encoded by the mRNA is detectable in the subject at least about 144 hours after administration. In some embodiments, the protein or the peptide encoded by the mRNA is detectable in the subject at least about 168 hours after administration.

In some embodiments, the protein or the peptide encoded by the mRNA is detectable in the subject at least about 1 day, 2 days, 3 days, 4 days, 5, days, 6 days, 7 days, 8 days, 9 days or 10 days after administration. In some embodiments, the protein or the peptide encoded by the mRNA is detectable in the subject at least about 1 day after administration. In some embodiments, the protein or the peptide encoded by the mRNA is detectable in the subject at least about 2 days after administration. In some embodiments, the protein or the peptide encoded by the mRNA is detectable in the subject at least about 3 days after administration. In some embodiments, the protein or the peptide encoded by the mRNA is detectable in the subject at least about 4 days after administration. In some embodiments, the protein or the peptide encoded by the mRNA is detectable in the subject at least about 5 days after administration. In some embodiments, the protein or the peptide encoded by the mRNA is detectable in the subject at least about 6 days after administration. In some embodiments, the protein or the peptide encoded by the mRNA is detectable in the subject at least about 7 days after administration. In some embodiments, the protein or the peptide encoded by the mRNA is detectable in the subject at least about 8 days after administration. In some embodiments, the protein or the peptide encoded by the mRNA is detectable in the subject at least about 9 days after administration. In some embodiments, the protein or the peptide encoded by the mRNA is detectable in the subject at least about 10 days after administration.

In some embodiments, the protein or the peptide encoded by the mRNA is detectable in the subject's circulation, liver, kidney, colon and/or rectum. at least about 6 hours, about 12 hours, about 18 hours, about 24 hours, about 36 hours, about 48 hours, about 60 hours, about 72 hours, about 96 hours, or about 120 hours after administration. In some embodiments, the protein or the peptide encoded by the mRNA is detectable in the subject's circulation, liver, kidney, colon and/or rectum at least about 6 hours after administration. In some embodiments, the protein or the peptide encoded by the mRNA is detectable in the subject's circulation, liver, kidney, colon and/or rectum at least about 12 hours after administration. In some embodiments, the protein or the peptide encoded by the mRNA is detectable in the subject's circulation, liver, kidney, colon and/or rectum at least about 18 hours after administration. In some embodiments, the protein or the peptide encoded by the mRNA is detectable in the subject's circulation, liver, kidney, colon and/or rectum at least about 24 hours after administration. In some embodiments, the protein or the peptide encoded by the mRNA is detectable in the subject's circulation, liver, kidney, colon and/or rectum at least about 36 hours after administration. In some embodiments, the protein or the peptide encoded by the mRNA is detectable in the subject's circulation, liver, kidney, colon and/or rectum at least about 48 hours after administration. In some embodiments, the protein or the peptide encoded by the mRNA is detectable in the subject's circulation, liver, kidney, colon and/or rectum at least about 60 hours after administration. In some embodiments, the protein or the peptide encoded by the mRNA is detectable in the subject's circulation, liver, kidney, colon and/or rectum at least about 72 hours after administration. In some embodiments, the protein or the peptide encoded by the mRNA is detectable in the subject's circulation, liver, kidney, colon and/or rectum at least about 96 hours after administration. In some embodiments, the protein or the peptide encoded by the mRNA is detectable in the subject's circulation, liver, kidney, colon and/or rectum at least about 120 hours after administration.

In some embodiments, the protein or the peptide encoded by the mRNA is detectable in the subject's circulation, liver, kidney, colon and/or rectum at least about 1 day, 2 days, 3 days, 4 days, 5, days, 6 days, 7 days, 8 days, 9 days or 10 days after administration. In some embodiments, the protein or the peptide encoded by the mRNA is detectable in the subject's circulation, liver, kidney, colon and/or rectum at least about 1 day after administration. In some embodiments, the protein or the peptide encoded by the mRNA is detectable in the subject's circulation, liver, kidney, colon and/or rectum at least about 2 days after administration. In some embodiments, the protein or the peptide encoded by the mRNA is detectable in the subject's circulation, liver, kidney, colon and/or rectum at least about 3 days after administration. In some embodiments, the protein or the peptide encoded by the mRNA is detectable in the subject's circulation, liver, kidney, colon and/or rectum at least about 4 days after administration. In some embodiments, the protein or the peptide encoded by the mRNA is detectable in the subject's circulation, liver, kidney, colon and/or rectum at least about 5 days after administration. In some embodiments, the protein or the peptide encoded by the mRNA is detectable in the subject's circulation, liver, kidney, colon and/or rectum at least about 6 days after administration. In some embodiments, the protein or the peptide encoded by the mRNA is circulation, liver, kidney, colon and/or rectum in the subject's circulation at least about 7 days after administration. In some embodiments, the protein or the peptide encoded by the mRNA is detectable in the subject's circulation, liver, kidney, colon and/or rectum at least about 8 days after administration. In some embodiments, the protein or the peptide encoded by the mRNA is detectable in the subject's circulation, liver, kidney, colon and/or rectum at least about 9 days after administration. In some embodiments, the protein or the peptide encoded by the mRNA is detectable in the subject's circulation, liver, kidney, colon and/or rectum at least about 10 days after administration.

In some embodiments, lipid-encapsulated mRNA is able to translocate following the rectal delivery (e.g., move intact by either active or passive means) to the systemic blood supply and subsequently reach different cells or target tissues.

Thus, in some aspects, the present invention provides a method of delivery of messenger RNA (mRNA) to a subject for in vivo production of a protein or a peptide in the subject, comprising administering to the subject by mucosal delivery a composition comprising an mRNA that encodes a protein or a peptide and is encapsulated within a lipid nanoparticle, and wherein the mRNA is detectable in the subject's circulation, liver, kidney, colon, and/or rectum at least about 24 hours, about 48 hours, about 72 hours, or about 96 hours after administration. In some embodiments, the in vivo production of the protein or the peptide occurs in the subject's circulation, liver, kidney, colon, and/or rectum. Accordingly, in some embodiments, the in vivo production of rectally delivered, lipid-encapsulated mRNA occurs in the subject's circulation. In some embodiments, the in vivo production of rectally delivered, lipid-encapsulated mRNA occurs in the subject's liver. In some embodiments, the in vivo production of rectally delivered, lipid-encapsulated mRNA occurs in the subject's kidney. In some embodiments, the in vivo production of rectally delivered, lipid-encapsulated mRNA occurs in the subject's colon. In some embodiments, the in vivo production of rectally delivered, lipid-encapsulated mRNA occurs in the subject's rectum.

In some embodiments, the mRNA is detectable in the subject's circulation, liver, kidney, colon, rectum, heart and/or spleen. In some embodiments, the mRNA is detectable in the subject's circulation. In some embodiments, the mRNA is detectable in the subject's liver. In some embodiments, the mRNA is detectable in the subject's kidney. In some embodiments, the mRNA is detectable in the subject's colon. In some embodiments, the mRNA is detectable in the subject's rectum. In some embodiments, the mRNA is detectable in the subject's heart. In some embodiments, the mRNA is detectable in the subject's spleen.

In some embodiments, the mRNA is detectable in the subject at least about 6 hours, about 12 hours, about 18 hours, about 24 hours, about 36 hours, about 48 hours, about 60 hours, about 72 hours, about 96 hours, or about 120 hours after administration. In some embodiments, the mRNA is detectable in the subject at least about 6 hours after administration. In some embodiments, the mRNA is detectable in the subject at least about 12 hours after administration. In some embodiments, the mRNA is detectable in the subject at least about 18 hours after administration. In some embodiments, the mRNA is detectable in the subject at least about 24 hours after administration. In some embodiments, the mRNA is detectable in the subject at least about 36 hours after administration. In some embodiments, the mRNA is detectable in the subject at least about 48 hours after administration. In some embodiments, the mRNA is detectable in the subject at least about 60 hours after administration. In some embodiments, the mRNA is detectable in the subject at least about 72 hours after administration. In some embodiments, the mRNA is detectable in the subject at least about 96 hours after administration. In some embodiments, the mRNA is detectable in the subject at least about 120 hours after administration.

In some embodiments, the mRNA is detectable in the subject at least about 1 day, 2 days, 3 days, 4 days, 5, days, 6 days, 7 days, 8 days, 9 days or 10 days after administration. In some embodiments, the mRNA is detectable in the subject at least about 1 day after administration. In some embodiments, the mRNA is detectable in the subject at least about 2 days after administration. In some embodiments, the mRNA is detectable in the subject at least about 3 days after administration. In some embodiments, the mRNA is detectable in the subject at least about 4 days after administration. In some embodiments, the mRNA is detectable in the subject at least about 5 days after administration. In some embodiments, the mRNA is detectable in the subject at least about 6 days after administration. In some embodiments, the mRNA is detectable in the subject at least about 7 days after administration. In some embodiments the mRNA is detectable in the subject at least about 8 days after administration. In some embodiments, the mRNA is detectable in the subject at least about 9 days after administration. In some embodiments, the mRNA is detectable in the subject at least about 10 days after administration.

### Composition of Formulation

The present invention provides, among other thing, effective compositions for delivering messenger RNA (mRNA) via rectal delivery. The composition described herein are suitable for delivery of mRNA via mucosal tissues, such as via the rectum.

In some embodiments, the composition comprises an mRNA that encodes a protein or a peptide, encapsulated within a lipid nanoparticle. In some embodiments, the composition further comprises a suppository component. In some embodiments, the composition further comprises a permeability enhancer.

Suppository

Compositions for rectal or vaginal (e.g., transvaginal) administration are typically suppositories which can be prepared by mixing compositions with suitable non-irritating excipients such as cocoa butter, polymers, hydrogel, glycerin, gelatin, polyethylene glycol or a suppository wax which are solid at ambient temperature but liquid at body temperature and therefore melt in the rectum or vaginal cavity and release the active ingredient. The types of material used depends on the type of suppository, the type of drug, and the conditions which the suppository will be stored.

The present invention provides, among other things, a suppository for effective delivery of mRNA encapsulated in lipid nanoparticle via a mucosal route, such as for example, rectal, vaginal, ocular, oral, and/or gastrointestinal route. In some embodiments, the lipid-encapsulated mRNA is delivered via the rectal or vaginal route. The suppository described herein, comprising a lipid nanoparticle and mRNA, is solid at room temperature and melts once administered rectally or vaginally. The melting of the suppository once placed in the rectum or vagina allows for the effective release of the mRNA-loaded lipid nanoparticles.

In some embodiments, the suppository is refrigerated prior to administration.

In some embodiments, the suppository softens or melts at about between 30 and 42° C. In some embodiments, the suppository softens or melts at about between 32 and 40° C. In some embodiments, the suppository softens or melts at about between 34 and 38° C. In some embodiments, the suppository softens or melts at about between 36 and 37° C. In some embodiments, the suppository softens or melts at about 36° C. In some embodiments, the suppository softens or melts at about 37° C.

In some embodiments the suppository softens or melts within 30 minutes once administered to the subject. In some embodiments the suppository softens or melts within 20 minutes once administered to the subject. In some embodiments the suppository softens or melts within 15 minutes once administered to the subject. In some embodiments the suppository softens or melts within 10 minutes once administered to the subject. In some embodiments the suppository softens or melts within 5 minutes once administered to the subject. In some embodiments the suppository softens or melts within 3 minutes once administered to the subject. In some embodiments the suppository softens or melts within 1 minute once administered to the subject.

As a non-limiting example, the formulations for rectal and/or vaginal administration may be prepared by mixing the drug with a suitable non-irritating excipient that is solid at ordinary temperatures but liquid at the rectal temperature and will therefore melt in the rectum and/or vagina to release the drug. Such materials include cocoa butter and polyethylene glycols.

A pharmaceutical composition for rectal or vaginal administration may comprise at least one inactive ingredient. Any or none of the inactive ingredients used may have been approved by the US Food and Drug Administration (FDA). A non-exhaustive list of inactive ingredients for use in pharmaceutical compositions for rectal or vaginal administration includes methylcellulose, hydroxyproplymethylcellulose, hydroxymethylcellulose, poloxamers, polyvinyl alcohols, polyvinyl pyrrolidones, polyacrylamides, polyethylene oxides, modified starches, adipic acid, alcohol, denatured, allantoin, anhydrous lactose, apricot kernel oil peg-6 esters, barium sulfate, beeswax, bentonite, benzoic acid, benzyl alcohol, butylated hydroxyanisole, butylated hydroxytoluene, calcium lactate, carbomer 934, carbomer 934p, cellulose, microcrystalline, ceteth-20, cetostearyl alcohol, cetyl alcohol, cetyl esters wax, cetyl palmitate, cholesterol, choleth, citric acid, citric acid monohydrate, coconut oil/palm kernel oil glycerides, hydrogenated, crospovidone, edetate disodium, ethylcelluloses, ethylene-vinyl acetate copolymer (28% vinyl acetate), ethylene-vinyl acetate copolymer (9% vinylacetate), fatty alcohols, fd&c yellow no. 5, gelatin, glutamic acid, dl-, glycerin, glyceryl isostearate, glyceryl monostearate, glyceryl stearate, guar gum, high density polyethylene, hydrogel polymer, hydrogenated palm oil, hypromellose 2208 (15000 mpa·s), hypromelloses, isopropyl myristate, lactic acid, lactic acid, dl-, lactose, lactose monohydrate, lactose, hydrous, lanolin, lanolin anhydrous, lecithin, lecithin, soybean, light mineral oil, magnesium aluminum silicate, magnesium aluminum silicate hydrate, magnesium stearate, methyl stearate, methylparaben, microcrystalline wax, mineral oil, nitric acid, octyldodecanol, peanut oil, peg 6-32 stearate/glycol stearate, peg-100 stearate, peg-120 glyceryl stearate, peg-2 stearate, peg-5 oleate, pegoxol 7 stearate, petrolatum, white, phenylmercuric acetate, phospholipon 90g, phosphoric acid, piperazine hexahydrate, poly(dimethylsiloxane/methylvinylsiloxane/methylhydrogensiloxane) dimethylvinyl or dimethylhydroxy or trimethyl endblocked, polycarbophil, polyester, polyethylene glycol 1000, polyethylene glycol 3350, polyethylene glycol 400, polyethylene glycol 4000, polyethylene glycol 6000, polyethylene glycol 8000, polyglyceryl-3 oleate, polyglyceryl-4 oleate, polyoxyl palmitate, polysorbate 20, polysorbate 60, polysorbate 80, polyurethane, potassium alum, potassium hydroxide, povidone k29/32, povidones, promulgen d, propylene glycol, propylene glycol monopalmitostearate, propylparaben, quaternium-15 cis-form, silicon dioxide, silicon dioxide, colloidal, silicone, sodium bicarbonate, sodium citrate, sodium hydroxide, sodium lauryl sulfate, sodium metabisulfite, sodium phosphate, dibasic, anhydrous, sodium phosphate, monobasic, anhydrous, sorbic acid, sorbitan monostearate, sorbitol, sorbitol solution, spermaceti, stannous 2-ethylhexanoate, starch, starch 1500, pregelatinized, starch, corn, stearamidoethyl diethylamine, stearic acid, stearyl alcohol, tartaric acid, dl-, tert-butylhydroquinone, tetrapropyl orthosilicate, trolamine, urea, vegetable oil, hydrogenated, wecobee fs, white ceresin wax and white wax.

In some embodiments, a gelatin water system is used in the formulations to keep the lipid nanoparticles intact. Gelatin aqueous solution is mucoadhesive and may assist in the contact of the suppository with the mucus membrane. Thus the presence of gelatin would assist the translocation of the mRNA-loaded lipid nanoparticles into systemic circulation. Furthermore, gelatin solution is a gel at room temperature which in turn prevents the mRNA-loaded nanoparticles from dripping out of the rectum. The gelatin further melts gradually at physiological temperature, enabling the mRNA loaded lipid nanoparticles to come in contact with the mucus membrane.

In some embodiments, the suppository comprises about 1% or more gelatin in water, 3% or more gelatin in water, 5% or more gelatin in water, 10% or more gelatin in water, 15% or more gelatin in water, 20% or more gelatin in water, 30% or more gelatin in water, 40% or more gelatin in water, 50% or more gelatin in water, 60% or more gelatin in water, 70% or more gelatin in water, 80% or more gelatin in water, 90% or more gelatin in water. In some embodiments, the suppository comprises about 1% or more gelatin in water. In some embodiments, the suppository comprises about 3% or more gelatin in water. In some embodiments, the suppository comprises about 5% or more gelatin in water. In some embodiments, the suppository comprises about 10% or more gelatin in water. In some embodiments, the suppository comprises about 15% or more gelatin in water. In some embodiments, the suppository comprises about 20% or more gelatin in water. In some embodiments, the suppository comprises about 30% or more gelatin in water. In some embodiments, the suppository comprises about 40% or more gelatin in water. In some embodiments, the suppository comprises about 50% or more gelatin in water. In some embodiments, the suppository comprises about 60% or more gelatin in water. In some embodiments, the suppository comprises about 70% or more gelatin in water. In some embodiments, the suppository comprises about 80% or more gelatin in water. In some embodiments, the suppository comprises about 90% or more gelatin in water.

In some embodiments the suppository does not adversely affect the integrity of the lipid nanoparticles.

In some embodiments, the composition does not comprise a lipid-based suppository component. In some embodiments, the composition comprises a lipid-based suppository component. In some embodiments, the lipid-based suppository component is cocoa butter, theobroma oil, synthetic fats or synthetic bases. In some embodiments, the lipid-based suppository component is cocoa butter, theobroma oil, synthetic fats or synthetic bases. In some embodiments, the lipid-based suppository component is cocoa butter. In some embodiments, the lipid-based suppository component is theobroma oil. In some embodiments, the lipid-based suppository component is synthetic fats. In some embodiments, the lipid-based suppository component synthetic bases.

In some embodiments, the composition comprises a water-based suppository component. In some embodiments, the water-based suppository component is selected from glycerin, gelatin or polyethylene glycol (PEG), or combinations thereof. In some embodiments, the water-based suppository component is glycerin. In some embodiments, the water-based suppository component is gelatin. In some embodiments, the water-based suppository component is polyethylene glycol (PEG). In some embodiments, the only water-based suppository component is gelatin.

In some embodiments, the suppository comprises glycerin and/or PEG. In some embodiments, the suppository comprises glycerin. In some embodiments, the suppository comprises PEG. In some embodiments, the suppository comprises less than about 10% glycerin. In some embodiments, the suppository comprises less than about 8% glycerin. In some embodiments, the suppository comprises less than about 6% glycerin. In some embodiments, the suppository comprises less than about 4% glycerin. In some embodiments, the suppository comprises less than about 2% glycerin. In some embodiments, the suppository comprises less than about 1% glycerin. In some embodiments, the suppository comprises less than about 0.1% glycerin. In some embodiments, the suppository comprises less than about 10% PEG. In some embodiments, the suppository comprises less than about 8% PEG. In some embodiments, the suppository comprises less than about 6% PEG. In some embodiments, the suppository comprises less than about 4% PEG. In some embodiments, the suppository comprises less than about 2% PEG. In some embodiments, the suppository comprises less than about 1% PEG. In some embodiments, the suppository comprises less than about 0.1% PEG.

In some embodiments, the suppository further comprises glycerol. In some embodiments, the amount of the glycerol present in the suppository does not destroy lipid nanoparticles. In some embodiments, the suppository does not comprise glycerol. In some embodiments, the suppository comprises less than about 30% glycerol. In some embodiments, the suppository comprises less than about 20% glycerol. In some embodiments, the suppository comprises less than about 15% glycerol. In some embodiments, the suppository comprises less than about 10% glycerol. In some embodiments, the suppository comprises less than about 8% glycerol. In some embodiments, the suppository comprises less than about 6% glycerol. In some embodiments, the suppository comprises less than about 4% glycerol. In some embodiments, the suppository comprises less than about 2% glycerol. In some embodiments, the suppository comprises less than about 1% glycerol. In some embodiments, the suppository comprises less than about 0.5% glycerol. In some embodiments, the suppository comprises less than about 0.1% glycerol.

The present invention provides, among other things, a suppository for rectal administration of mRNA. In some embodiments, the suppository comprises mRNA encapsulated within a lipid nanoparticle, wherein the mRNA encodes a protein or a peptide and gelatin.

The suppository described herein is formulated to hold a various concentrations of mRNA, with a size that allows convenient and non-invasive administration via rectal or vaginal delivery. Such non-invasive routes of delivery unexpectedly provide an effective means to conveniently deliver therapeutic compositions.

In some embodiments, the composition comprises 0.25 mg/mL or greater mRNA, 0.5 mg/mL or greater mRNA, 0.75 mg/mL or greater mRNA, or 1 mg/mL or greater mRNA. In some embodiments, composition comprises 0.1 mg/mL or greater mRNA. In some embodiments, composition comprises 0.25 mg/mL or greater mRNA. In some embodiments, the composition comprises 0.5 mg/mL or greater mRNA. In some embodiments, the composition comprises 0.75 mg/mL or greater mRNA. In some embodiments, the composition comprises 1 mg/mL or greater mRNA. In some embodiments, composition comprises 2 mg/mL or greater mRNA. In some embodiments, the composition comprises 2.5 mg/mL or greater mRNA. In some embodiments, the composition comprises 5 mg/mL or greater mRNA.

In some embodiments, the composition comprises 0.5 mg or greater mRNA, 0.75 mg or greater mRNA, 1 mg or greater mRNA, 1.25 mg or greater mRNA, 1.5 mg or greater mRNA, or 1.75 mg or greater mRNA. In some embodiments, the composition comprises 0.1 mg or greater mRNA. In some embodiments, the composition comprises 0.25 mg or greater mRNA. In some embodiments, the composition comprises 0.5 mg or greater mRNA. In some embodiments, the composition comprises 0.75 mg or greater mRNA. In some embodiments, the composition comprises 1 mg or greater mRNA. In some embodiments, the composition comprises 1.25 mg or greater mRNA. In some embodiments, the composition comprises 1.5 mg or greater mRNA. In some embodiments, the composition comprises 1.75 mg or greater mRNA. In some embodiments, the composition comprises 2 mg or greater mRNA. In some embodiments, the composition comprises 2.5 mg or greater mRNA. In some embodiments, the composition comprises 5 mg or greater mRNA.

In some embodiments, the composition is formulated for a suppository of about 3 grams, about 2 grams, or about 1 gram. In some embodiments, the composition is formulated for a suppository of about 20 grams. In some embodiments, the composition is formulated for a suppository of about 10 grams. In some embodiments, the composition is formulated for a suppository of about 5 grams. In some embodiments, the composition is formulated for a suppository of about 3 grams. In some embodiments, the composition is formulated for a suppository of about 2 grams. In some embodiments, the composition is formulated for a suppository of about 1 gram. In some embodiments, the composition is formulated for a suppository of about 0.5 grams.

In some embodiments, the composition is formulated for a suppository having a volume of about 2.0 mL, about 3.5 mL, about 7.5 mL, or about 10.0 mL. In some embodiments, the composition is formulated for a suppository having a volume of about 1.0 mL. In some embodiments, the composition is formulated for a suppository having a volume of about 2.0 mL. In some embodiments, the composition is formulated for a suppository having a volume of about 2.5 mL. In some embodiments, the composition is formulated for a suppository having a volume of about 3.0 mL. In some embodiments, the composition is formulated for a suppository having a volume of about 3.5 mL. In some embodiments, the composition is formulated for a suppository having a volume of about 4.0 mL. In some embodiments, the composition is formulated for a suppository having a volume of about 5.0 mL. In some embodiments, the composition is formulated for a suppository having a volume of about 7.5 mL. In some embodiments, the composition is formulated for a suppository having a volume of about 10.0 mL. In some embodiments, the composition is formulated for a suppository having a volume of about 12.5 mL. In some embodiments, the composition is formulated for a suppository having a volume of about 15.0 mL. In some embodiments, the composition is formulated for a suppository having a volume of about 17.5 mL. In some embodiments, the composition is formulated for a suppository having a volume of about 20.0 mL.

Permeability Enhancers

To improve bioavailability of drugs with poor absorption across mucosal routes (e.g., rectal, vaginal, ocular, oral, or gastrointestinal), penetration or permeability enhancers have be used. In some embodiments for rectal or vaginal administration, the suppository further comprises a permeability enhancer. In some embodiments the suppository does not comprise a permeability enhancer. In some embodiments, the permeability enhancer does not adversely affect the integrity of the lipid nanoparticle.

In some embodiments, the permeability enhancer is selected from bile salts, surfactants, fatty acids and derivatives, glycerides, chelators, salicylates, or polymers. In some embodiments, the permeability enhancer is a bile salt. In some embodiments, the permeability enhancer is a fatty acid and derivatives thereof. In some embodiments, the permeability enhancer is glycerides. In some embodiments, the permeability enhancer is a chelator. In some embodiments, the permeability enhancer is a salicylate. In some embodiments, the permeability enhancer is a polymer.

In some embodiments, the fatty acids and derivatives are selected from sorbitan laurate, sodium caprate, sucrose, palitate, lauroyl choline, sodium myristate, or palmitoyl carnitine. In some embodiments, the fatty acids and derivatives include sorbitan laurate. In some embodiments, the fatty acids and derivatives include sodium caprate. In some embodiments, the fatty acids and derivatives are sucrose. In some embodiments, the fatty acids and derivatives include palitate. In some embodiments, the fatty acids and derivatives include lauroyl choline. In some embodiments, the fatty acids and derivatives include sodium myristate. In some embodiments, the fatty acids and derivatives include palmitoyl carnitine.

In some embodiments, the permeability enhancer is a form of caprate. In some embodiments, the caprate-based permeability enhancer is sodium caprate.

In some embodiments, the permeability enhancer include cholates. In some embodiments, the permeability enhancer is citric acid. In some embodiments, the permeability enhancer is ethylenediaminetetraacetic acid (EDTA). In some embodiments, the permeability enhancer is oleic acid. In some embodiments, the permeability enhancer is caprates. In some embodiments, the permeability enhancer is sulfactants. In some embodiments, the permeability enhancer is sodium dodecyl sulfate (SDS). In some embodiments, the permeability enhancer is Cremophor®. In some embodiments, the permeability enhancer is Tween® 80, In some embodiments, the permeability enhancer is Labrasol®. In some embodiments, the permeability enhancer is self-microemulsifying drug delivery system (SMEDDS). In some embodiments, the permeability enhancer is natural bioenhancers. In some embodiments, the permeability enhancer is allicin. In some embodiments, the permeability enhancer is piperine. In some embodiments, the permeability enhancer is curcumin. In some embodiments, the permeability enhancer is quercetin.

Several different formulations of the lipid-encapsulated mRNA composition have been devised to facilitate delivery to a subject, including administering a permeability enhancer prior to the administering the composition comprising mRNA. The administrating the permeability enhancer facilitates transfer of the mRNA-loaded lipid nanoparticles from colon to systemic circulation.

In some embodiments, the subject is first administered a permeability enhancer prior to the administering of the composition comprising mRNA. In some embodiments, the permeability enhancer is administered to the subject about 30 minutes, about 1 hour, about 2.5 hours, about 5 hours, or about 12 hours prior to administering the composition comprising mRNA. In some embodiments, the permeability enhancer is administered to the subject about 1 minute prior to administering the composition comprising mRNA. In some embodiments, the permeability enhancer is administered to the subject about 3 minutes prior to administering the composition comprising mRNA. In some embodiments, the permeability enhancer is administered to the subject about 5 minutes prior to administering the composition comprising mRNA. In some embodiments, the permeability enhancer is administered to the subject about 10 minutes prior to administering the composition comprising mRNA. In some embodiments, the permeability enhancer is administered to the subject about 15 minutes prior to administering the composition comprising mRNA. In some embodiments, the permeability enhancer is administered to the subject about 20 minutes prior to administering the composition comprising mRNA. In some embodiments, the permeability enhancer is administered to the subject about 25 minutes prior to administering the composition comprising mRNA. In some embodiments, the permeability enhancer is administered to the subject about 30 minutes prior to administering the composition comprising mRNA. In some embodiments, the permeability enhancer is administered to the subject about 45 minutes prior to administering the composition comprising mRNA. In some embodiments, the permeability enhancer is administered to the subject about 1 hour prior to administering the composition comprising mRNA. In some embodiments, the permeability enhancer is administered to the subject about 1.5 hours prior to administering the composition comprising mRNA. In some embodiments, the permeability enhancer is administered to the subject about 2 hours prior to administering the composition comprising mRNA. In some embodiments, the permeability enhancer is administered to the subject about 2.5 hours prior to administering the composition comprising mRNA. In some embodiments, the permeability enhancer is administered to the subject about 5 hours prior to administering the composition comprising mRNA. In some embodiments, the permeability enhancer is administered to the subject about 12 hours prior to administering the composition comprising mRNA. In some embodiments, the permeability enhancer is administered to the subject about 18 hours prior to administering the composition comprising mRNA. In some embodiments, the permeability enhancer is administered to the subject about 24 hours prior to administering the composition comprising mRNA.

In some embodiments, the permeability enhancer is administered to the subject simultaneously with the composition comprising mRNA. In some embodiments, the subject is administered a permeability enhancer post administering of the composition comprising mRNA. In some embodiments, the subject is administered a permeability enhancer about 5 minutes post administering of the composition comprising mRNA. In some embodiments, the subject is administered a permeability enhancer about 10 minutes post administering of the composition comprising mRNA. In some embodiments, the subject is administered a permeability enhancer about 15 minutes post administering of the composition comprising mRNA. In some embodiments, the subject is administered a permeability enhancer about 20 minutes post administering of the composition comprising mRNA. In some embodiments, the subject is administered a permeability enhancer about 30 minutes post administering of the composition comprising mRNA. In some embodiments, the subject is administered a permeability enhancer about 45 minutes post administering of the composition comprising mRNA. In some embodiments, the subject is administered a permeability enhancer about 1 hour post administering of the composition comprising mRNA. In some embodiments, the subject is administered a permeability enhancer about 2 hours post administering of the composition comprising mRNA. In some embodiments, the subject is administered a permeability enhancer about 2.5 hours post administering of the composition comprising mRNA. In some embodiments, the subject is administered a permeability enhancer about 5 hours post administering of the composition comprising mRNA. In some embodiments, the subject is administered a permeability enhancer about 12 hours post administering of the composition comprising mRNA. In some embodiments, the subject is administered a permeability enhancer about 18 hours post administering of the composition comprising mRNA. In some embodiments, the subject is administered a permeability enhancer about 24 hours post administering of the composition comprising mRNA.

mRNA Synthesis

mRNAs according to the present invention may be synthesized according to any of a variety of known methods. Various methods are described in published U.S. Application No. US 2018/0258423, and can be used to practice the present invention, all of which are incorporated herein by reference. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

In some embodiments, a suitable mRNA sequence is an mRNA sequence encoding a protein or a peptide. In some embodiments, a suitable mRNA sequence is codon optimized for efficient expression human cells. In some embodiments, a suitable mRNA sequence is naturally-occurring or a wild-type sequence. In some embodiments, a suitable mRNA sequence encodes a protein or a peptide that contains one or mutations in amino acid sequence.

The present invention may be used to deliver mRNAs of a variety of lengths. In some embodiments, the present invention may be used to deliver in vitro synthesized mRNA of or greater than about 0.5 kb, 1 kb, 1.5 kb, 2 kb, 2.5 kb, 3 kb, 3.5 kb, 4 kb, 4.5 kb, 5 kb 6 kb, 7 kb, 8 kb, 9 kb, 10 kb, 11 kb, 12 kb, 13 kb, 14 kb, 15 kb, 20 kb, 30 kb, 40 kb, or 50 kb in length. In some embodiments, the present invention may be used to deliver in vitro synthesized mRNA ranging from about 1-20 kb, about 1-15 kb, about 1-10 kb, about 5-20 kb, about 5-15 kb, about 5-12 kb, about 5-10 kb, about 8-20 kb, or about 8-50 kb in length.

In some embodiments, for the preparation of mRNA according to the invention, a DNA template is transcribed in vitro. A suitable DNA template typically has a promoter, for example a T3, T7 or SP6 promoter, for in vitro transcription, followed by desired nucleotide sequence for desired mRNA and a termination signal.

Nucleotides

Various naturally-occurring or modified nucleosides may be used to produce mRNA according to the present invention. In some embodiments, an mRNA is or comprises naturally-occurring nucleosides (or unmodified nucleotides; e.g., adenosine, guanosine, cytidine, uridine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, pseudouridine, (e.g., N-1-methyl-pseudouridine), 2-thiouridine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, a suitable mRNA may contain backbone modifications, sugar modifications and/or base modifications. For example, modified nucleotides may include, but not be limited to, modified purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, the mRNA comprises one or more nonstandard nucleotide residues. The nonstandard nucleotide residues may include, e.g., 5-methylcytidine (“5mC”), pseudouridine (“ψU”), and/or 2-thio-uridine (“2sU”). See, e.g., U.S. Pat. No. 8,278,036 or WO 2011/012316 for a discussion of such residues and their incorporation into mRNA. The mRNA may be RNA, which is defined as RNA in which 25% of U residues are 2-thio-uridine and 25% of C residues are 5-methylcytidine. Teachings for the use of RNA are disclosed US Patent Publication US 2012/0195936 and international publication WO 2011/012316, both of which are hereby incorporated by reference in their entirety. The presence of nonstandard nucleotide residues may render an mRNA more stable and/or less immunogenic than a control mRNA with the same sequence but containing only standard residues. In further embodiments, the mRNA may comprise one or more nonstandard nucleotide residues chosen from isocytosine, pseudoisocytosine, 5-bromouracil, 5-propynyluracil, 6-aminopurine, 2-aminopurine, inosine, diaminopurine and 2-chloro-6-aminopurine cytosine, as well as combinations of these modifications and other nucleobase modifications. Some embodiments may further include additional modifications to the furanose ring or nucleobase. Additional modifications may include, for example, sugar modifications or substitutions (e.g., one or more of a 2′-O-alkyl modification, a locked nucleic acid (LNA)). In some embodiments, the RNAs may be complexed or hybridized with additional polynucleotides and/or peptide polynucleotides (PNA). In some embodiments where the sugar modification is a 2′-O-alkyl modification, such modification may include, but are not limited to a 2′-deoxy-2′-fluoro modification, a 2′-O-methyl modification, a 2′-O-methoxyethyl modification and a 2′-deoxy modification. In some embodiments, any of these modifications may be present in 0-100% of the nucleotides—for example, more than 0%, 1%, 10%, 25%, 50%, 75%, 85%, 90%, 95%, or 100% of the constituent nucleotides individually or in combination.

In some embodiments, mRNAs may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g., cytidine 5′-O-(1-thiophosphate)), boranophosphates, positively charged guanidinium groups etc., which means by replacing the phosphodiester linkage by other anionic, cationic or neutral groups.

In some embodiments, mRNAs may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

Post-Synthesis Processing

Typically, a 5′ cap and/or a 3′ tail may be added after the synthesis. The presence of the cap is important in providing resistance to nucleases found in most eukaryotic cells. The presence of a “tail” serves to protect the mRNA from exonuclease degradation.

A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published U.S. Application No. US 2016/0032356 and published U.S. Application No. US 2018/0125989, which are incorporated herein by reference.

Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly A or poly C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly (A) and poly (C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

As described herein, the addition of the 5′ cap and/or the 3′ tail facilitates the detection of abortive transcripts generated during in vitro synthesis because without capping and/or tailing, the size of those prematurely aborted mRNA transcripts can be too small to be detected. Thus, in some embodiments, the 5′ cap and/or the 3′ tail are added to the synthesized mRNA before the mRNA is tested for purity (e.g., the level of abortive transcripts present in the mRNA). In some embodiments, the 5′ cap and/or the 3′ tail are added to the synthesized mRNA before the mRNA is purified as described herein. In other embodiments, the 5′ cap and/or the 3′ tail are added to the synthesized mRNA after the mRNA is purified as described herein.

mRNA synthesized according to the present invention may be used without further purification. In particular, mRNA synthesized according to the present invention may be used without a step of removing shortmers. In some embodiments, mRNA synthesized according to the present invention may be further purified. Various methods may be used to purify mRNA synthesized according to the present invention. For example, purification of mRNA can be performed using centrifugation, filtration and/or chromatographic methods. In some embodiments, the synthesized mRNA is purified by ethanol precipitation or filtration or chromatography, or gel purification or any other suitable means. In some embodiments, the mRNA is purified by HPLC. In some embodiments, the mRNA is extracted in a standard phenol: chloroform: isoamyl alcohol solution, well known to one of skill in the art. In some embodiments, the mRNA is purified using Tangential Flow Filtration. Suitable purification methods include those described in published U.S. Application No. US 2016/0040154, published U.S. Application No. US 2015/0376220, published U.S. Application No. US 2018/0251755, published U.S. Application No. US 2018/0251754, U.S. Provisional Application No. 62/757,612 filed on Nov. 8, 2018, and U.S. Provisional Application No. 62/891,781 filed on Aug. 26, 2019, all of which are incorporated by reference herein and may be used to practice the present invention.

In some embodiments, the mRNA is purified before capping and tailing. In some embodiments, the mRNA is purified after capping and tailing. In some embodiments, the mRNA is purified both before and after capping and tailing.

In some embodiments, the mRNA is purified either before or after or both before and after capping and tailing, by centrifugation.

In some embodiments, the mRNA is purified either before or after or both before and after capping and tailing, by filtration.

In some embodiments, the mRNA is purified either before or after or both before and after capping and tailing, by Tangential Flow Filtration (TFF).

In some embodiments, the mRNA is purified either before or after or both before and after capping and tailing by chromatography.

Characterization of Purified mRNA

The mRNA composition described herein is substantially free of contaminants comprising short abortive RNA species, long abortive RNA species, double-stranded RNA (dsRNA), residual plasmid DNA, residual in vitro transcription enzymes, residual solvent and/or residual salt.

The mRNA composition described herein has a purity of about between 60% and about 100%. Accordingly, in some embodiments, the purified mRNA has a purity of about 60%. In some embodiments, the purified mRNA has a purity of about 65%. In some embodiments, the purified mRNA has a purity of about 70%. In some embodiments, the purified mRNA has a purity of about 75%. In some embodiments, the purified mRNA has a purity of about 80%. In some embodiments, the purified mRNA has a purity of about 85%. In some embodiments, the purified mRNA has a purity of about 90%. In some embodiments, the purified mRNA has a purity of about 91%. In some embodiments, the purified mRNA has a purity of about 92%. In some embodiments, the purified mRNA has a purity of about 93%. In some embodiments, the purified mRNA has a purity of about 94%. In some embodiments, the purified mRNA has a purity of about 95%. In some embodiments, the purified mRNA has a purity of about 96%. In some embodiments, the purified mRNA has a purity of about 97%. In some embodiments, the purified mRNA has a purity of about 98%. In some embodiments, the purified mRNA has a purity of about 99%. In some embodiments, the purified mRNA has a purity of about 100%.

In some embodiments, the mRNA composition described herein has less than 10%, less than 9%, less than 8%, less than 7%, less than 6%, less than 5%, less than 4%, less than 3%, less than 2%, less than 1%, less than 0.5%, and/or less than 0.1% impurities other than full-length mRNA. The impurities include IVT contaminants, e.g., proteins, enzymes, DNA templates, free nucleotides, residual solvent, residual salt, double-stranded RNA (dsRNA), prematurely aborted RNA sequences (“shortmers” or “short abortive RNA species”), and/or long abortive RNA species. In some embodiments, the purified mRNA is substantially free of process enzymes.

In some embodiments, the residual plasmid DNA in the purified mRNA of the present invention is less than about 1 pg/mg, less than about 2 pg/mg, less than about 3 pg/mg, less than about 4 pg/mg, less than about 5 pg/mg, less than about 6 pg/mg, less than about 7 pg/mg, less than about 8 pg/mg, less than about 9 pg/mg, less than about 10 pg/mg, less than about 11 pg/mg, or less than about 12 pg/mg. Accordingly, the residual plasmid DNA in the purified mRNA is less than about 1 pg/mg. In some embodiments, the residual plasmid DNA in the purified mRNA is less than about 2 pg/mg. In some embodiments, the residual plasmid DNA in the purified mRNA is less than about 3 pg/mg. In some embodiments, the residual plasmid DNA in the purified mRNA is less than about 4 pg/mg. In some embodiments, the residual plasmid DNA in the purified mRNA is less than about 5 pg/mg. In some embodiments, the residual plasmid DNA in the purified mRNA is less than about 6 pg/mg. In some embodiments, the residual plasmid DNA in the purified mRNA is less than about 7 pg/mg. In some embodiments, the residual plasmid DNA in the purified mRNA is less than about 8 pg/mg. In some embodiments, the residual plasmid DNA in the purified mRNA is less than about 9 pg/mg. In some embodiments, the residual plasmid DNA in the purified mRNA is less than about 10 pg/mg. In some embodiments, the residual plasmid DNA in the purified mRNA is less than about 11 pg/mg. In some embodiments, the residual plasmid DNA in the purified mRNA is less than about 12 pg/mg.

In some embodiments, a method according to the invention removes more than about 90%, 95%, 96%, 97%, 98%, 99% or substantially all prematurely aborted RNA sequences (also known as “shortmers”). In some embodiments, mRNA composition is substantially free of prematurely aborted RNA sequences. In some embodiments, mRNA composition contains less than about 5% (e.g., less than about 4%, 3%, 2%, or 1%) of prematurely aborted RNA sequences. In some embodiments, mRNA composition contains less than about 1% (e.g., less than about 0.9%, 0.8%, 0.7%, 0.6%, 0.5%, 0.4%, 0.3%, 0.2%, or 0.1%) of prematurely aborted RNA sequences. In some embodiments, mRNA composition undetectable prematurely aborted RNA sequences as determined by, e.g., high-performance liquid chromatography (HPLC) (e.g., shoulders or separate peaks), ethidium bromide, Coomassie staining, capillary electrophoresis or Glyoxal gel electrophoresis (e.g., presence of separate lower band). As used herein, the term “shortmers”, “short abortive RNA species”, “prematurely aborted RNA sequences” or “long abortive RNA species” refers to any transcripts that are less than full-length. In some embodiments, “shortmers”, “short abortive RNA species”, or “prematurely aborted RNA sequences” are less than 100 nucleotides in length, less than 90, less than 80, less than 70, less than 60, less than 50, less than 40, less than 30, less than 20, or less than 10 nucleotides in length. In some embodiments, shortmers are detected or quantified after adding a 5′-cap, and/or a 3′-poly A tail. In some embodiments, prematurely aborted RNA transcripts comprise less than 15 bases (e.g., less than 14, 13, 12, 11, 10, 9, 8, 7, 6, 5, 4, or 3 bases). In some embodiments, the prematurely aborted RNA transcripts contain about 8-15, 8-14, 8-13, 8-12, 8-11, or 8-10 bases.

In some embodiments, a purified mRNA of the present invention is substantially free of enzyme reagents used in in vitro synthesis including, but not limited to, T7 RNA polymerase, DNAse I, pyrophosphatase, and/or RNAse inhibitor. In some embodiments, a purified mRNA according to the present invention contains less than about 5% (e.g., less than about 4%, 3%, 2%, or 1%) of enzyme reagents used in in vitro synthesis including. In some embodiments, a purified mRNA contains less than about 1% (e.g., less than about 0.9%, 0.8%, 0.7%, 0.6%, 0.5%, 0.4%, 0.3%, 0.2%, or 0.1%) of enzyme reagents used in in vitro synthesis including. In some embodiments, a purified mRNA contains undetectable enzyme reagents used in in vitro synthesis including as determined by, e.g., silver stain, gel electrophoresis, high-performance liquid chromatography (HPLC), ultra-performance liquid chromatography (UPLC), and/or capillary electrophoresis, ethidium bromide and/or Coomassie staining.

In various embodiments, a purified mRNA of the present invention maintains high degree of integrity. As used herein, the term “mRNA integrity” generally refers to the quality of mRNA after purification. mRNA integrity may be determined using methods well known in the art, for example, by RNA agarose gel electrophoresis. In some embodiments, mRNA integrity may be determined by banding patterns of RNA agarose gel electrophoresis. In some embodiments, a purified mRNA of the present invention shows little or no banding compared to reference band of RNA agarose gel electrophoresis. In some embodiments, a purified mRNA of the present invention has an integrity greater than about 95% (e.g., greater than about 96%, 97%, 98%, 99% or more). In some embodiments, a purified mRNA of the present invention has an integrity greater than 98%. In some embodiments, a purified mRNA of the present invention has an integrity greater than 99%. In some embodiments, a purified mRNA of the present invention has an integrity of approximately 100%.

In some embodiments, the purified mRNA is assessed for one or more of the following characteristics: appearance, identity, quantity, concentration, presence of impurities, microbiological assessment, pH level and activity. In some embodiments, acceptable appearance includes a clear, colorless solution, essentially free of visible particulates. In some embodiments, the identity of the mRNA is assessed by sequencing methods. In some embodiments, the concentration is assessed by a suitable method, such as UV spectrophotometry. In some embodiments, a suitable concentration is between about 90% and 110% nominal (0.9-1.1 mg/mL).

In some embodiments, assessing the purity of the mRNA includes assessment of mRNA integrity, assessment of residual plasmid DNA, and assessment of residual solvent. In some embodiments, acceptable levels of mRNA integrity are assessed by agarose gel electrophoresis. The gels are analyzed to determine whether the banding pattern and apparent nucleotide length is consistent with an analytical reference standard. Additional methods to assess RNA integrity include, for example, assessment of the purified mRNA using capillary gel electrophoresis (CGE). In some embodiments, acceptable purity of the purified mRNA as determined by CGE is that the purified mRNA composition has no greater than about 55% long abortive/degraded species. In some embodiments, residual plasmid DNA is assessed by methods in the art, for example by the use of qPCR. In some embodiments, less than 10 pg/mg (e.g., less than 10 pg/mg, less than 9 pg/mg, less than 8 pg/mg, less than 7 pg/mg, less than 6 pg/mg, less than 5 pg/mg, less than 4 pg/mg, less than 3 pg/mg, less than 2 pg/mg, or less than 1 pg/mg) is an acceptable level of residual plasmid DNA. In some embodiments, acceptable residual solvent levels are not more than 10,000 ppm, 9,000 ppm, 8,000 ppm, 7,000 ppm, 6,000 ppm, 5,000 ppm, 4,000 ppm, 3,000 ppm, 2,000 ppm, 1,000 ppm. Accordingly, in some embodiments, acceptable residual solvent levels are not more than 10,000 ppm. In some embodiments, acceptable residual solvent levels are not more than 9,000 ppm. In some embodiments, acceptable residual solvent levels are not more than 8,000 ppm. In some embodiments, acceptable residual solvent levels are not more than 7,000 ppm. In some embodiments, acceptable residual solvent levels are not more than 6,000 ppm. In some embodiments, acceptable residual solvent levels are not more than 5,000 ppm. In some embodiments, acceptable residual solvent levels are not more than 4,000 ppm. In some embodiments, acceptable residual solvent levels are not more than 3,000 ppm. In some embodiments, acceptable residual solvent levels are not more than 2,000 ppm. In some embodiments, acceptable residual solvent levels are not more than 1,000 ppm.

In some embodiments, microbiological tests are performed on the purified mRNA, which include, for example, assessment of bacterial endotoxins. In some embodiments, bacterial endotoxins are <0.5 EU/mL, <0.4 EU/mL, <0.3 EU/mL, <0.2 EU/mL or <0.1 EU/mL. Accordingly, in some embodiments, bacterial endotoxins in the purified mRNA are <0.5 EU/mL. In some embodiments, bacterial endotoxins in the purified mRNA are <0.4 EU/mL. In some embodiments, bacterial endotoxins in the purified mRNA are <0.3 EU/mL. In some embodiments, bacterial endotoxins in the purified mRNA are <0.2 EU/mL. In some embodiments, bacterial endotoxins in the purified mRNA are <0.2 EU/mL. In some embodiments, bacterial endotoxins in the purified mRNA are <0.1 EU/mL. In some embodiments, the purified mRNA has not more than 1 CFU/10 mL, 1 CFU/25 mL, 1 CFU/50 mL, 1 CFU/75 mL, or not more than 1 CFU/100 mL. Accordingly, in some embodiments, the purified mRNA has not more than 1 CFU/10 mL. In some embodiments, the purified mRNA has not more than 1 CFU/25 mL. In some embodiments, the purified mRNA has not more than 1 CFU/50 mL. In some embodiments, the purified mRNA has not more than 1 CFR/75 mL. In some embodiments, the purified mRNA has 1 CFU/100 mL.

In some embodiments, the pH of the purified mRNA is assessed. In some embodiments, acceptable pH of the purified mRNA is between 5 and 8. Accordingly, in some embodiments, the purified mRNA has a pH of about 5. In some embodiments, the purified mRNA has a pH of about 6. In some embodiments, the purified mRNA has a pH of about 7. In some embodiments, the purified mRNA has a pH of about 7. In some embodiments, the purified mRNA has a pH of about 8.

In some embodiments, the translational fidelity of the purified mRNA is assessed. The translational fidelity can be assessed by various methods and include, for example, transfection and Western blot analysis. Acceptable characteristics of the purified mRNA includes banding pattern on a Western blot that migrates at a similar molecular weight as a reference standard.

In some embodiments, the purified mRNA is assessed for conductance. In some embodiments, acceptable characteristics of the purified mRNA include a conductance of between about 50% and 150% of a reference standard.

The purified mRNA is also assessed for Cap percentage and for PolyA tail length. In some embodiments, an acceptable Cap percentage includes Cap1, % Area: NLT90. In some embodiments, an acceptable PolyA tail length is about 100-1500 nucleotides (e.g., 100, 150, 200, 250, 300, 350, 400, 450, 500, 550, 600, 650, 700, 750, 800, 850, 900, 950, and 1000, 1100, 1200, 1300, 1400, or 1500 nucleotides).

In some embodiments, the purified mRNA is also assessed for any residual PEG. In some embodiments, the purified mRNA has less than between 10 ng PEG/mg of purified mRNA and 1000 ng PEG/mg of mRNA. Accordingly, in some embodiments, the purified mRNA has less than about 10 ng PEG/mg of purified mRNA. In some embodiments, the purified mRNA has less than about 100 ng PEG/mg of purified mRNA. In some embodiments, the purified mRNA has less than about 250 ng PEG/mg of purified mRNA. In some embodiments, the purified mRNA has less than about 500 ng PEG/mg of purified mRNA. In some embodiments, the purified mRNA has less than about 750 ng PEG/mg of purified mRNA. In some embodiments, the purified mRNA has less than about 1000 ng PEG/mg of purified mRNA.

Various methods of detecting and quantifying mRNA purity are known in the art. For example, such methods include, blotting, capillary electrophoresis, chromatography, fluorescence, gel electrophoresis, HPLC, silver stain, spectroscopy, ultraviolet (UV), or UPLC, or a combination thereof. In some embodiments, mRNA is first denatured by a Glyoxal dye before gel electrophoresis (“Glyoxal gel electrophoresis”). In some embodiments, synthesized mRNA is characterized before capping or tailing. In some embodiments, synthesized mRNA is characterized after capping and tailing.

### Delivery Vehicles

According to the present invention, mRNA or MCNA encoding a protein or a peptide (e.g., a full length, fragment, or portion of a protein or a peptide) as described herein may be delivered as naked RNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

In some embodiments, mRNAs or MCNAs encoding at least one protein or peptide may be delivered via a single delivery vehicle. In some embodiments, mRNAs or MCNAs encoding at least one protein or peptide may be delivered via one or more delivery vehicles each of a different composition. In some embodiments, the one or more mRNAs and/or MCNAs are encapsulated within the same lipid nanoparticles. In some embodiments, the one or more mRNAs are encapsulated within separate lipid nanoparticles.

According to various embodiments, suitable delivery vehicles include, but are not limited to polymer based carriers, such as polyethyleneimine (PEI), lipid nanoparticles and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags. Also contemplated is the use of bionanocapsules and other viral capsid proteins assemblies as a suitable transfer vehicle. (Hum. Gene Ther. 2008 September; 19(9):887-95).

Liposomal Delivery Vehicles

In some embodiments, a suitable delivery vehicle is a liposomal delivery vehicle, e.g., a lipid nanoparticle. As used herein, liposomal delivery vehicles, e.g., lipid nanoparticles, are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.). In the context of the present invention, a liposomal delivery vehicle typically serves to transport a desired nucleic acid (e.g., mRNA or MCNA) to a target cell or tissue. In some embodiments, a nanoparticle delivery vehicle is a liposome. In some embodiments, a liposome comprises one or more cationic lipids, one or more non-cationic lipids, one or more cholesterol-based lipids, or one or more PEG-modified lipids. In some embodiments, a liposome comprises no more than three distinct lipid components. In some embodiments, one distinct lipid component is a sterol-based cationic lipid.

Cationic Lipids

As used herein, the phrase “cationic lipids” refers to any of a number of lipid species that have a net positive charge at a selected pH, such as physiological pH.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

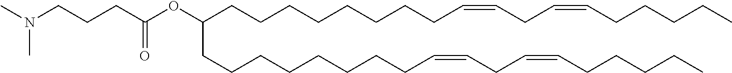 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

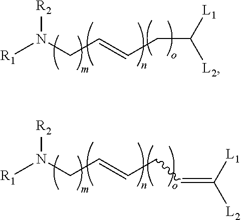

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z,18Z)-N,N-dimethyl-6-(9Z,12Z)-octadeca-9,12-dien-1-yl)tetracosa-15,18-dien-1-amine (“HGT5000”), having a compound structure of:

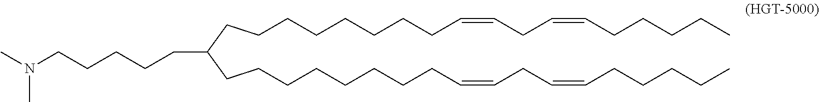 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z,18Z)-N,N-dimethyl-6-((9Z,12Z)-octadeca-9,12-dien-1-yl)tetracosa-4,15,18-trien-1-amine (“HGT5001”), having a compound structure of:

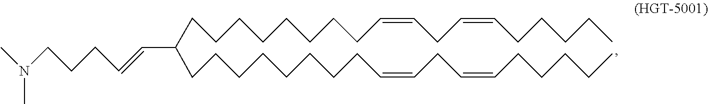 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)-N,N-dimethyl-6-((9Z,12Z)-octadeca-9,12-dien-1-yl)tetracosa-5,15,18-trien-1-amine (“HGT5002”), having a compound structure of:

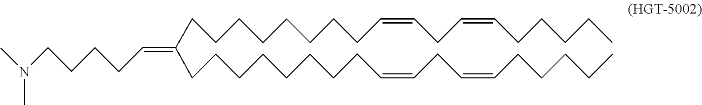 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

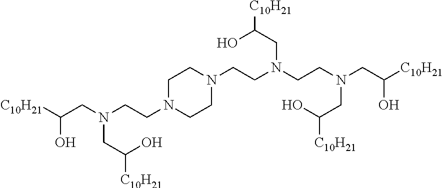

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

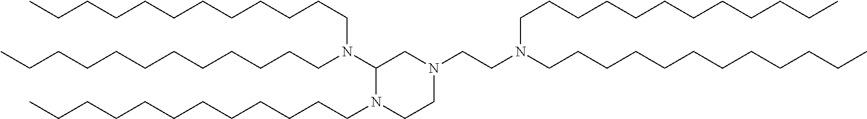 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

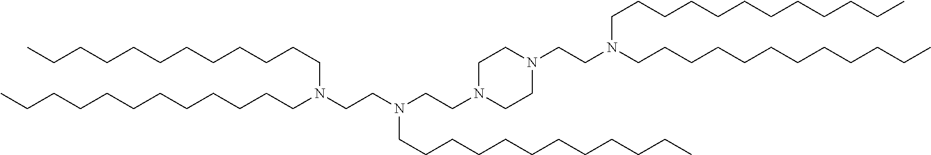 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

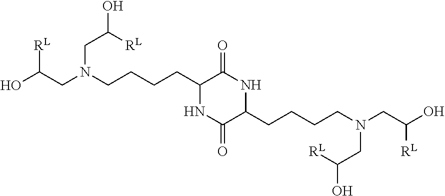

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

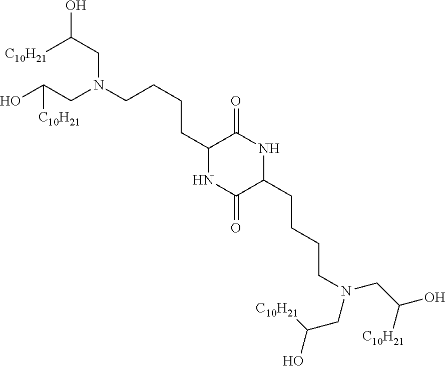

and pharmaceutically acceptable salts thereof. In certain embodiments, the compositions and methods of the present invention include a cationic lipid having a compound structure of:

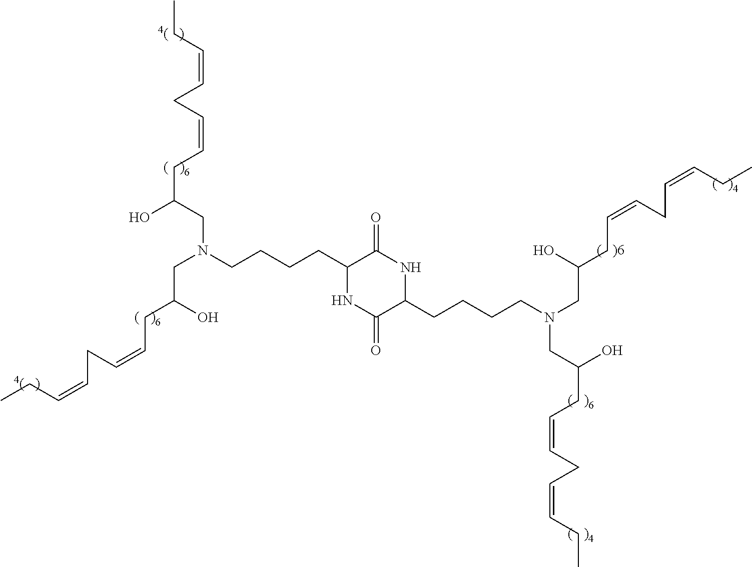

and pharmaceutically acceptable salts thereof. In certain embodiments, the compositions and methods of the present invention include a cationic lipid having a compound structure of:

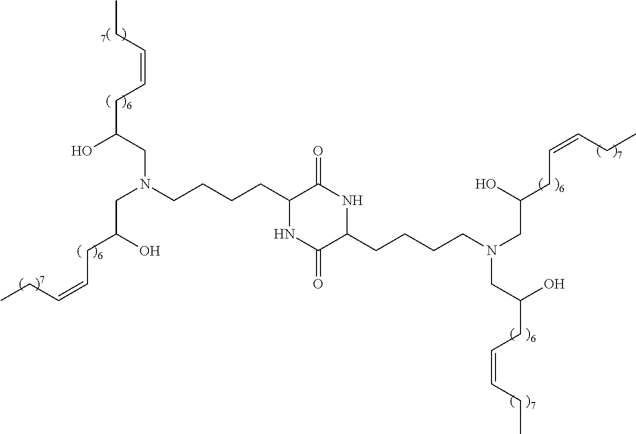

and pharmaceutically acceptable salts thereof. In certain embodiments, the compositions and methods of the present invention include a cationic lipid having a compound structure of:

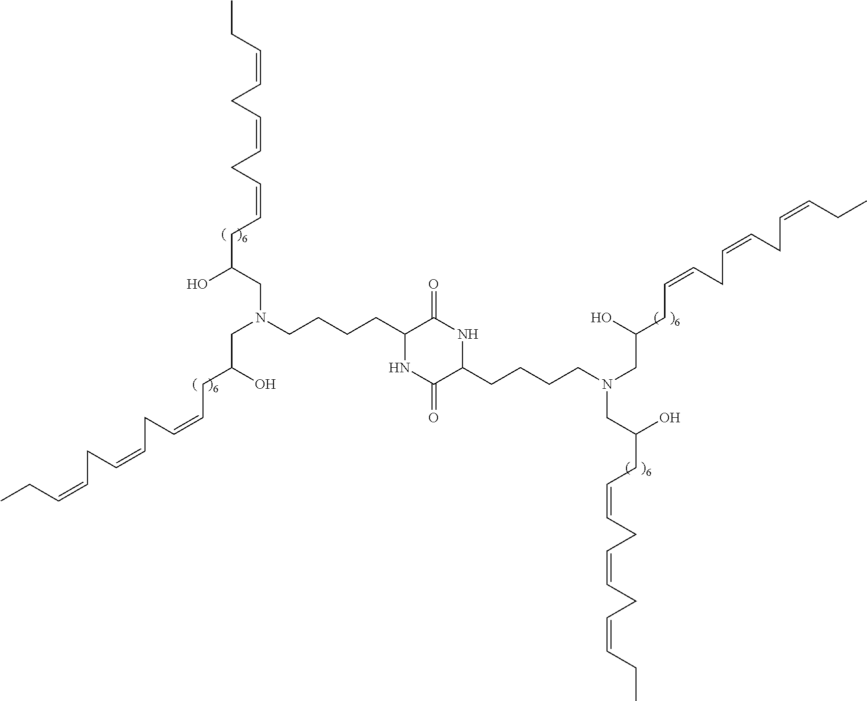

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

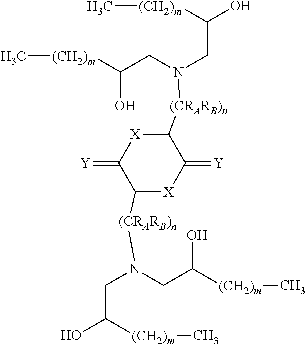

or a pharmaceutically acceptable salt thereof, wherein each X independently is O or S; each Y independently is O or S; each m independently is 0 to 20; each n independently is 1 to 6; each R_{A} is independently hydrogen, optionally substituted C₁-50 alkyl, optionally substituted C2-50 alkenyl, optionally substituted C₂-50 alkynyl, optionally substituted C₃-10 carbocyclyl, optionally substituted 3-14 membered heterocyclyl, optionally substituted C₆₋₁₄ aryl, optionally substituted 5-14 membered heteroaryl or halogen; and each RB is independently hydrogen, optionally substituted C₁-50 alkyl, optionally substituted C₂-50 alkenyl, optionally substituted C₂-50 alkynyl, optionally substituted C₃₋₁₀ carbocyclyl, optionally substituted 3-14 membered heterocyclyl, optionally substituted C₆₋₁₄ aryl, optionally substituted 5-14 membered heteroaryl or halogen. In certain embodiments, the compositions and methods of the present invention include a cationic lipid, “Target 23”, having a compound structure of:

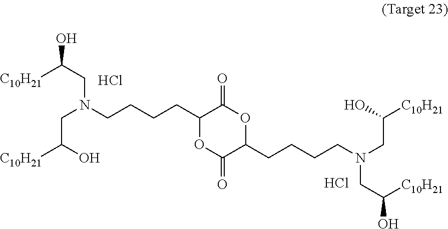

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

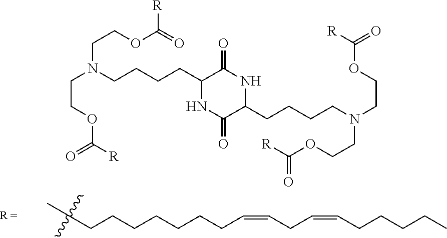

or a pharmaceutically acceptable salt thereof. In some embodiments, the compositions and methods of the present invention include a cationic lipid having the compound structure:

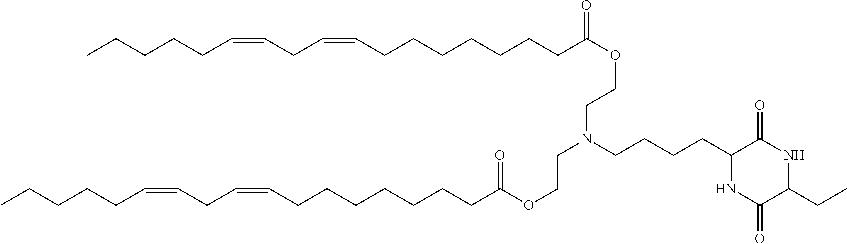 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

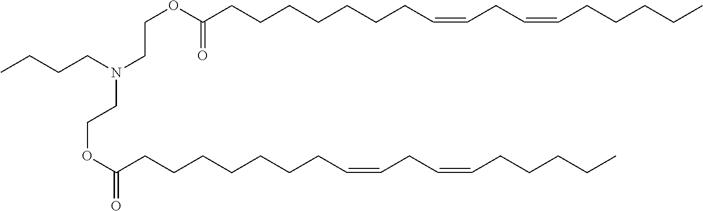 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include cationic lipids as described in U.S. Provisional Patent Application Ser. No. 62/758,179, which is incorporated herein by reference. In some embodiments, the compositions and methods of the present invention include a cationic lipid of the following formula:

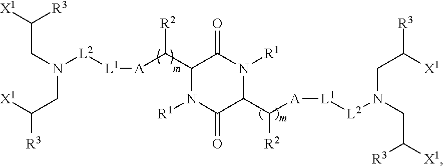

or a pharmaceutically acceptable salt thereof, wherein each R¹ and R² is independently H or C₁-C₆ aliphatic; each m is independently an integer having a value of 1 to 4; each A is independently a covalent bond or arylene; each L¹ is independently an ester, thioester, disulfide, or anhydride group; each L² is independently C₂-C₁₀ aliphatic; each X¹ is independently H or OH; and each R³ is independently C₆-C₂₀ aliphatic. In some embodiments, the compositions and methods of the present invention include a cationic lipid of the following formula:

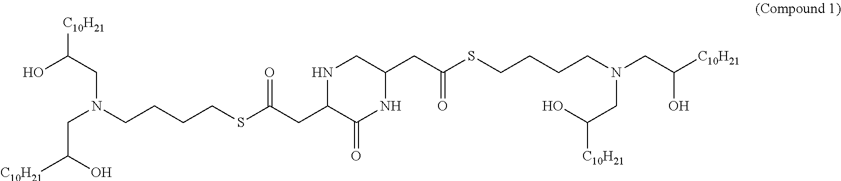

or a pharmaceutically acceptable salt thereof. In some embodiments, the compositions and methods of the present invention include a cationic lipid of the following formula:

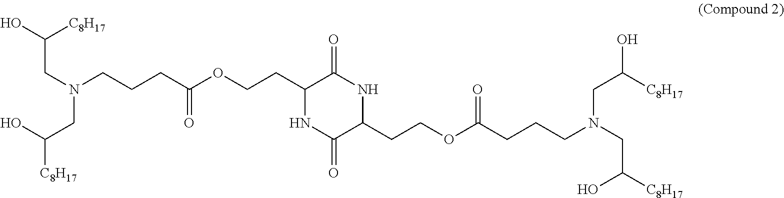

or a pharmaceutically acceptable salt thereof. In some embodiments, the compositions and methods of the present invention include a cationic lipid of the following formula:

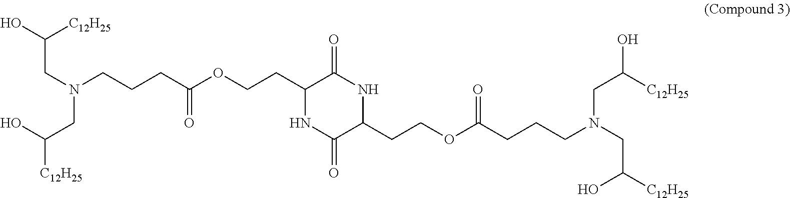

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

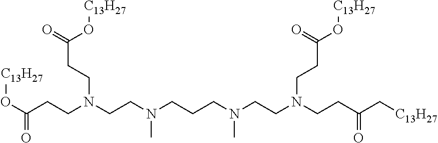

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

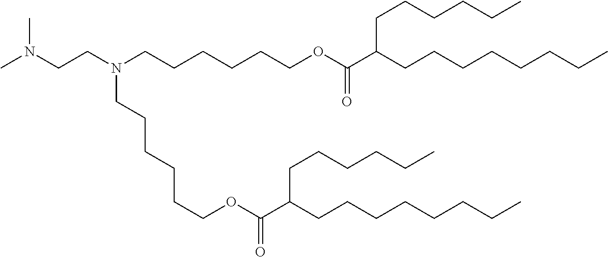 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

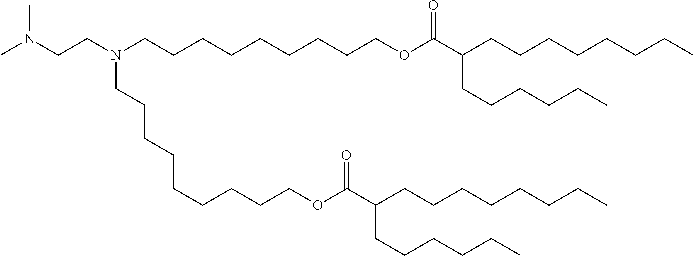 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

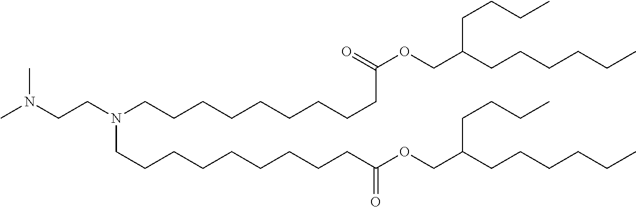 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

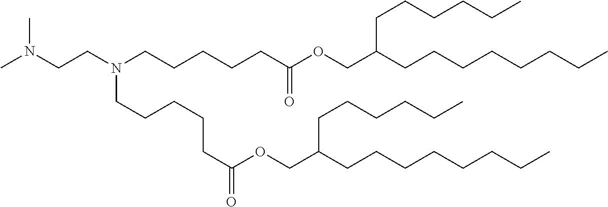 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

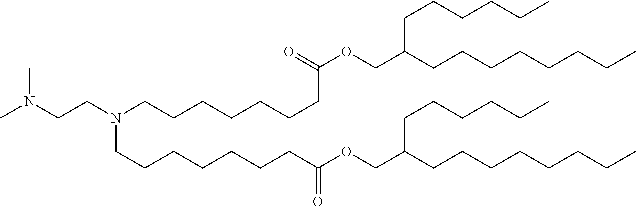 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

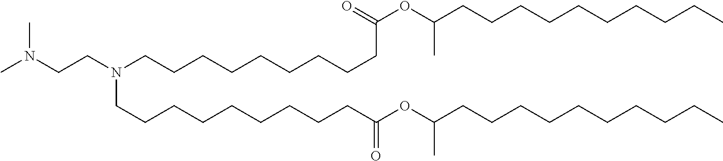 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

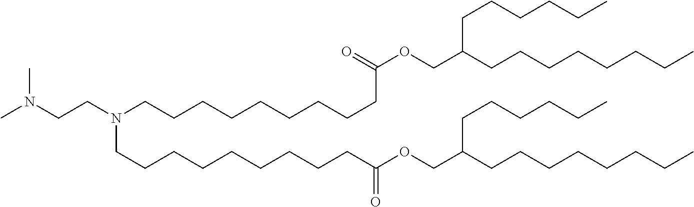 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

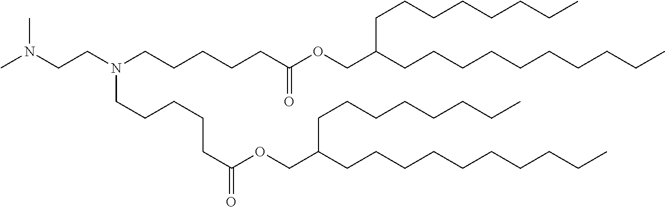 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

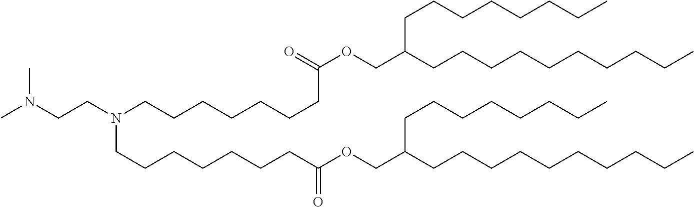 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

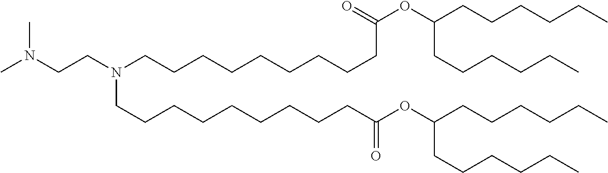 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

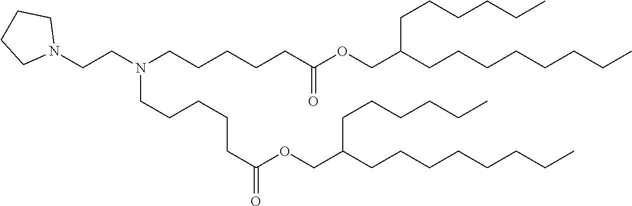 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

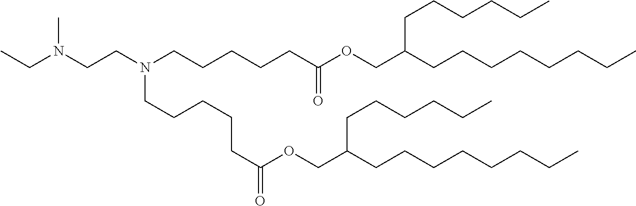 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

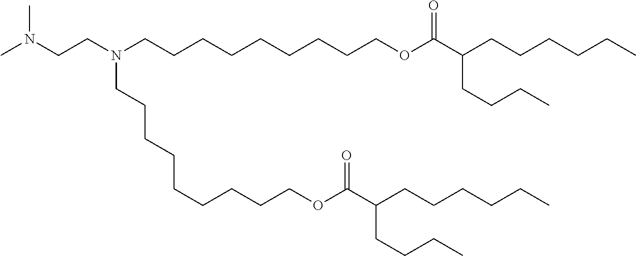 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

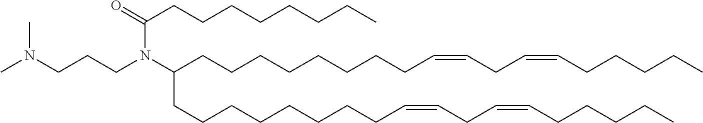 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

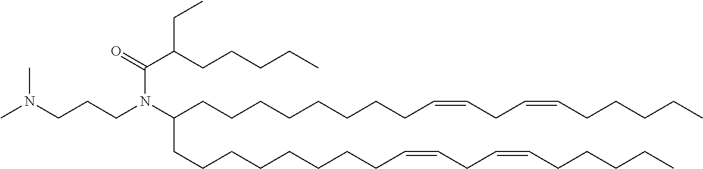 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

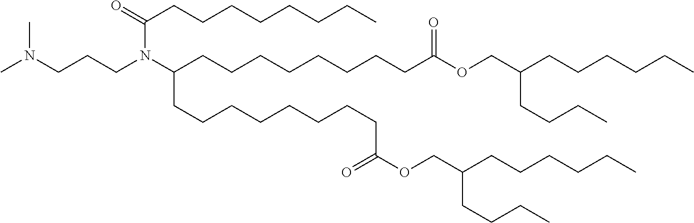 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

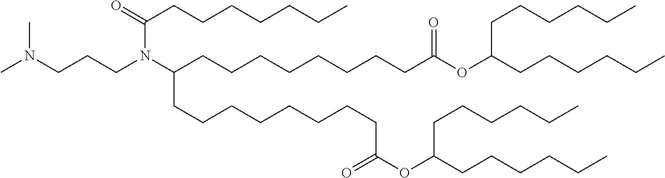 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

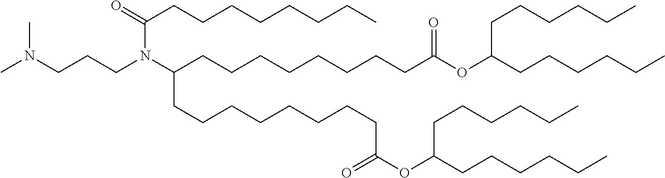 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

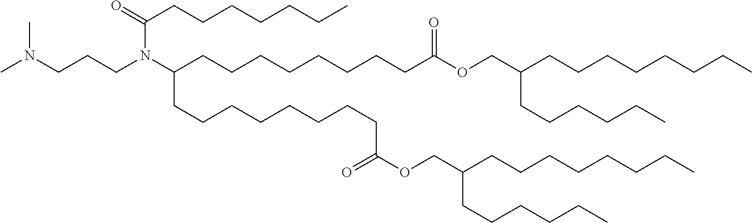 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

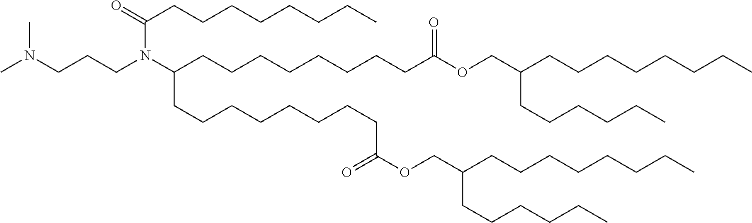 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

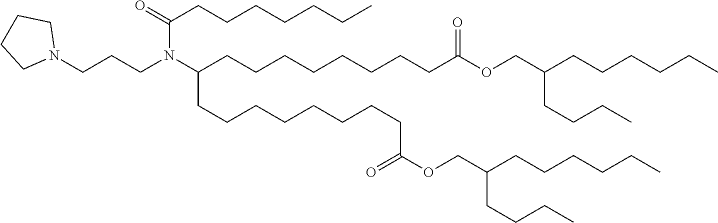 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

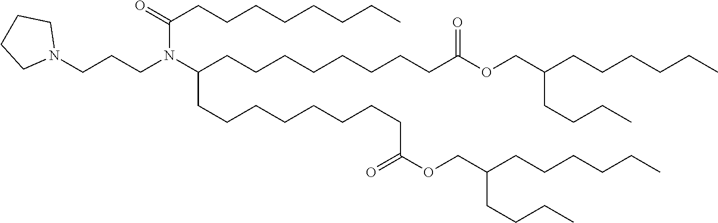 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

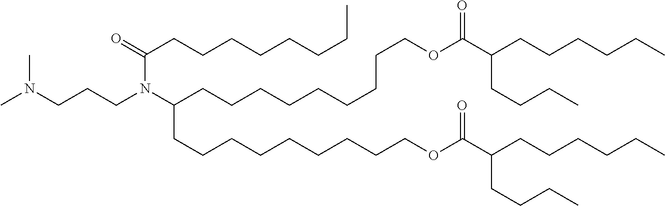 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

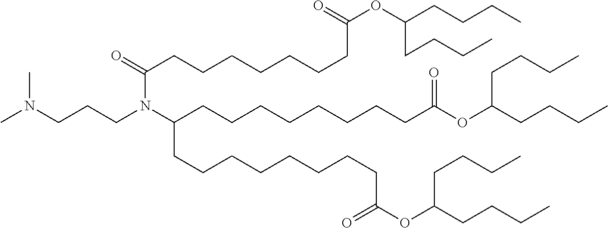 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

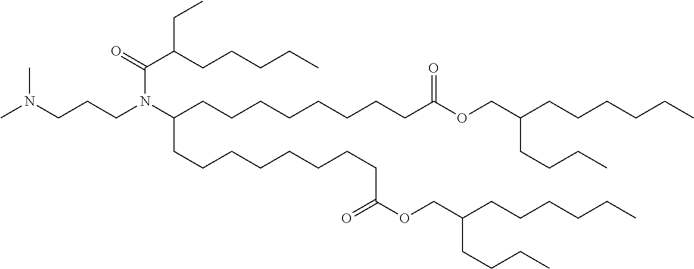 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

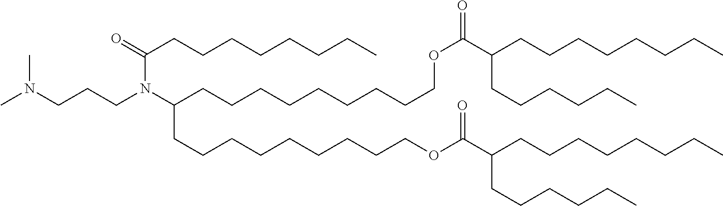 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

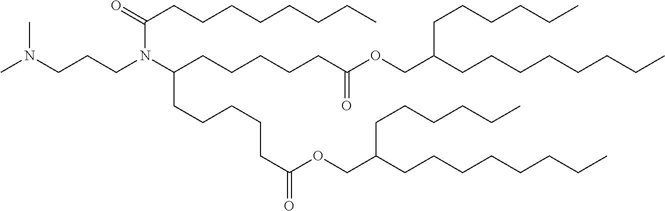 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

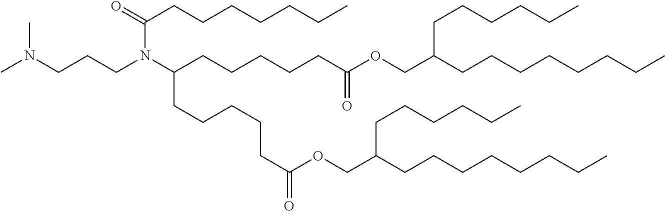 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

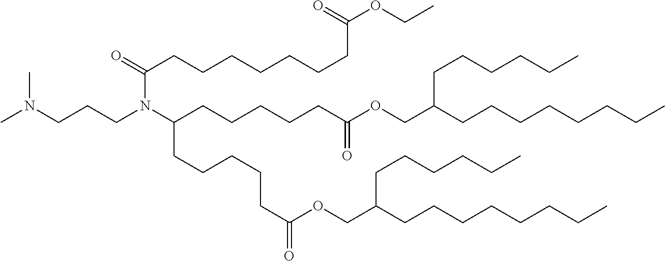 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

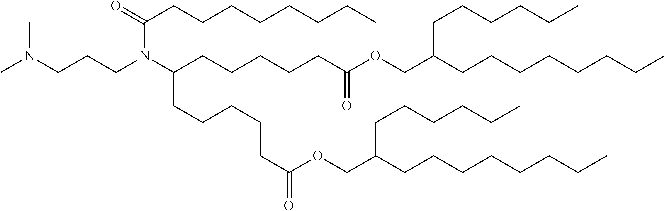 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

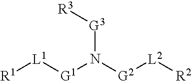

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

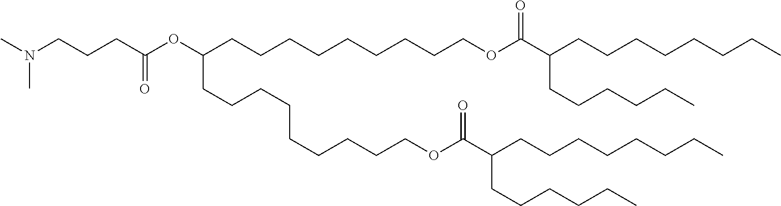 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

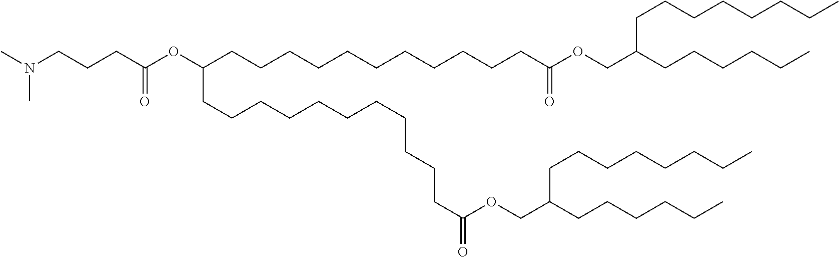 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

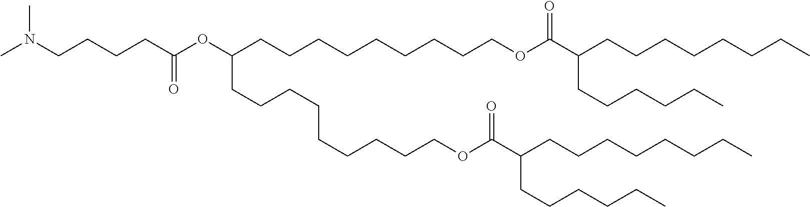 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

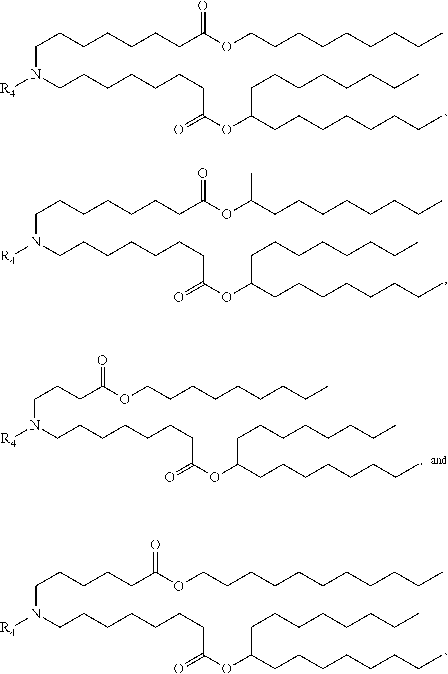

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

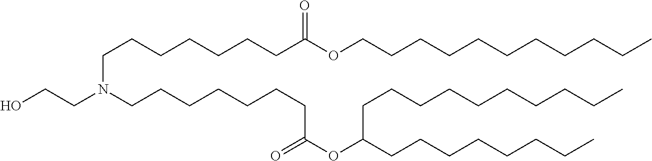 [SMILES: CCCCCCCCCCCOC(=O)CCCCCCCN(CCO)CCCCCCCC(=O)OC(CCCCCCCC)CCCCCCCCCC]

and pharmaceutically acceptable salts thereof. In certain embodiments, the compositions and methods of the present invention include a cationic lipid having a compound structure of:

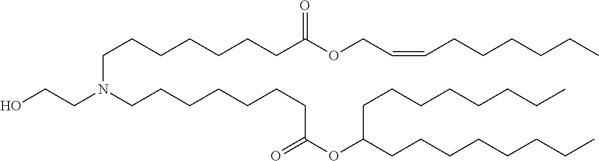 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

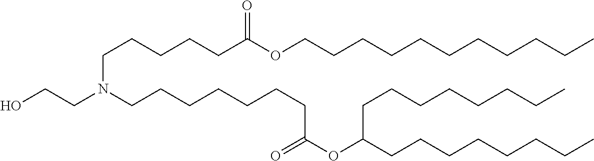 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

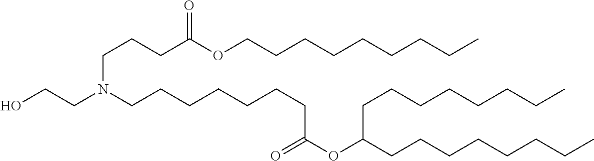 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

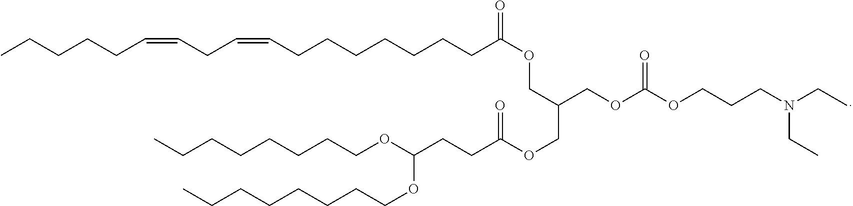 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In certain embodiments, the compositions and methods of the present invention include a cationic lipid having a compound structure of:

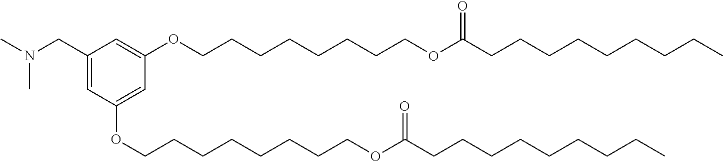 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

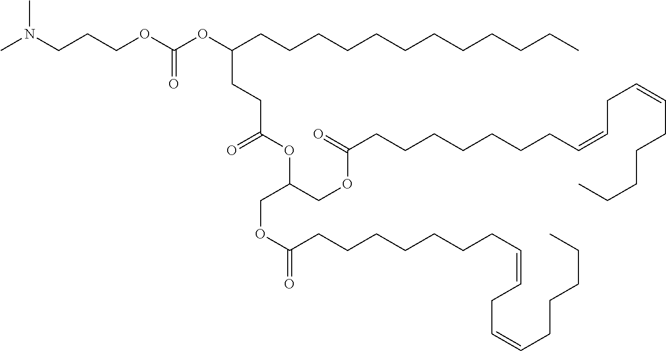 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

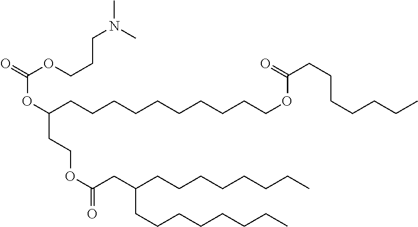 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

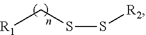

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

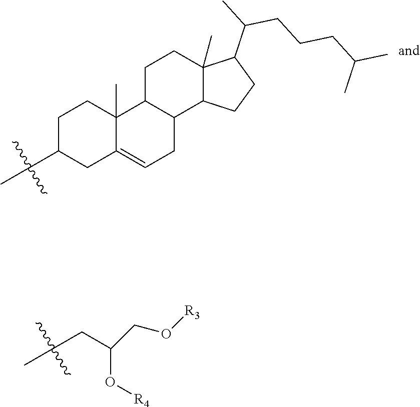

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

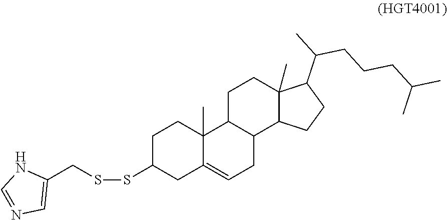 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002,” having a compound structure of:

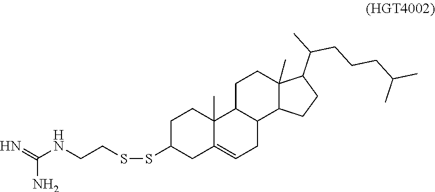 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003,” having a compound structure of:

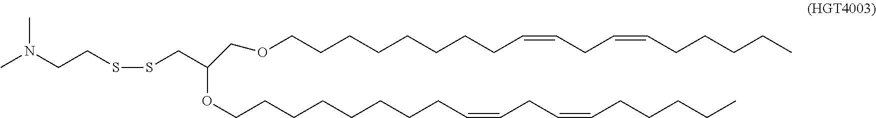 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004,” having a compound structure of:

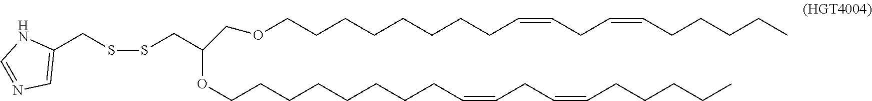 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005,” having a compound structure of:

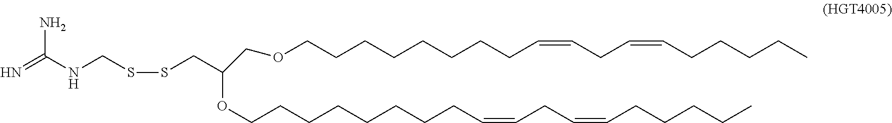 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

Other suitable cationic lipids for use in the compositions and methods of the present invention include cleavable cationic lipids as described in International Application No. PCT/US2019/032522, and incorporated herein by reference. In certain embodiments, the compositions and methods of the present invention include a cationic lipid that is any of general formulas or any of structures (1a)-(21a) and (1b)-(21b) and (22)-(237) described in International Application No. PCT/US2019/032522. In certain embodiments, the compositions and methods of the present invention include a cationic lipid that has a structure according to Formula (I′),

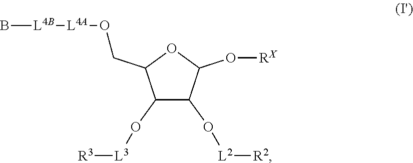

wherein:
R^{X} is independently —H, -L¹-R¹, or -L^{5A}-L^{5B}-B′;
each of L¹, L², and L³ is independently a covalent bond, —C(O)—, —C(O)O—, —C(O)S—, or —C(O)NR^{L}—;
each L^{4A} and L^{SA} is independently —C(O)—, —C(O)O—, or —C(O)NR^{L}—;
each L^{4B} and L^{SB} is independently C₁-C₂₀ alkylene; C₂-C₂₀ alkenylene; or C₂-C₂₀ alkynylene;
each B and B′ is NR⁴R⁵ or a 5- to 10-membered nitrogen-containing heteroaryl;
each R¹, R², and R³ is independently C₆-C₃₀ alkyl, C₆-C₃₀ alkenyl, or C₆-C₃₀ alkynyl;
each R⁴ and R⁵ is independently hydrogen, C₁-C₁₀ alkyl; C₂-C₁₀ alkenyl; or C₂-C₁₀ alkynyl; and
each R^{L} is independently hydrogen, C₁-C₂₀ alkyl, C₂-C₂₀ alkenyl, or C₂-C₂₀ alkynyl.
   In certain embodiments, the compositions and methods of the present invention include a cationic lipid that is Compound (139) of International Application No. PCT/US2019/032522, having a compound structure of:

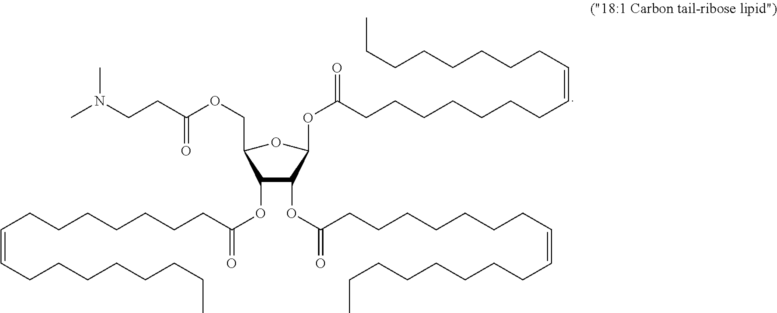

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylammonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethyl-1-(cis,cis-9′,1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N,N-dimethyl-3-[(9Z,12Z)-octadeca-9,12-dien-1-yloxy]propane-1-amine (“Octyl-CLinDMA”); (2R)-2-((8-[(3beta)-cholest-5-en-3-yloxy]octyl)oxy)-N,N-dimethyl-3-[(9Z,12Z)-octadeca-9,12-dien-1-yloxy]propan-1-amine (“Octyl-CLinDMA (2R)”); (2S)-2-((8-[(3P)-cholest-5-en-3-yloxy]octyl)oxy)-N, fsl-dimethyh3-[(9Z,12Z)-octadeca-9,12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoleyl-4-dimethylaminoethyl-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d][1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

Non-Cationic/Helper Lipids

In some embodiments, the liposomes contain one or more non-cationic (“helper”) lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, a non-cationic lipid is a neutral lipid, i.e., a lipid that does not carry a net charge in the conditions under which the composition is formulated and/or administered.

In some embodiments, such non-cationic lipids may be used alone, but are preferably used in combination with other lipids, for example, cationic lipids.

In some embodiments, a non-cationic lipid may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a molar ratio (mol %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 mol %, greater than about 10 mol %, greater than about 20 mol %, greater than about 30 mol %, or greater than about 40 mol %. In some embodiments, the percentage total non-cationic lipids in a liposome may be greater than about 5 mol %, greater than about 10 mol %, greater than about 20 mol %, greater than about 30 mol %, or greater than about 40 mol %. In some embodiments, the percentage of non-cationic lipid in a liposome is no more than about 5 mol %, no more than about 10 mol %, no more than about 20 mol %, no more than about 30 mol %, or no more than about 40 mol %. In some embodiments, the percentage total non-cationic lipids in a liposome may be no more than about 5 mol %, no more than about 10 mol %, no more than about 20 mol %, no more than about 30 mol %, or no more than about 40 mol %.

In some embodiments, a non-cationic lipid may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, total non-cationic lipids may be present in a weight ratio (wt %) of about 5% to about 90%, about 5% to about 70%, about 5% to about 50%, about 5% to about 40%, about 5% to about 30%, about 10% to about 70%, about 10% to about 50%, or about 10% to about 40% of the total lipids present in a composition. In some embodiments, the percentage of non-cationic lipid in a liposome may be greater than about 5 wt %, greater than about 10 wt %, greater than about 20 wt %, greater than about 30 wt %, or greater than about 40 wt %. In some embodiments, the percentage total non-cationic lipids in a liposome may be greater than about 5 wt %, greater than about 10 wt %, greater than about 20 wt %, greater than about 30 wt %, or greater than about 40 wt %. In some embodiments, the percentage of non-cationic lipid in a liposome is no more than about 5 wt %, no more than about 10 wt %, no more than about 20 wt %, no more than about 30 wt %, or no more than about 40 wt %. In some embodiments, the percentage total non-cationic lipids in a liposome may be no more than about 5 wt %, no more than about 10 wt %, no more than about 20 wt %, no more than about 30 wt %, or no more than about 40 wt %.

Cholesterol-Based Lipids

In some embodiments, the liposomes comprise one or more cholesterol-based lipids. For example, suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or imidazole cholesterol ester (ICE), which has the following structure,

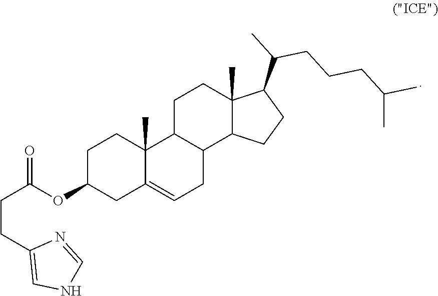 [SMILES: CC(C)CCCC(C)[C@H]1CCC2C3CC=C4C[C@@H](OC(=O)CCc5c[nH]cn5)CC[C@]4(C)C3CC[C@@]21C]

In embodiments, a cholesterol-based lipid is cholesterol.

In some embodiments, the cholesterol-based lipid may comprise a molar ratio (mol %) of about 1% to about 30%, or about 5% to about 20% of the total lipids present in a liposome. In some embodiments, the percentage of cholesterol-based lipid in the lipid nanoparticle may be greater than about 5 mol %, greater than about 10 mol %, greater than about 20 mol %, greater than about 30 mol %, or greater than about 40 mol %. In some embodiments, the percentage of cholesterol-based lipid in the lipid nanoparticle may be no more than about 5 mol %, no more than about 10 mol %, no more than about 20 mol %, no more than about 30 mol %, or no more than about 40 mol %.

In some embodiments, a cholesterol-based lipid may be present in a weight ratio (wt %) of about 1% to about 30%, or about 5% to about 20% of the total lipids present in a liposome. In some embodiments, the percentage of cholesterol-based lipid in the lipid nanoparticle may be greater than about 5 wt %, greater than about 10 wt %, greater than about 20 wt %, greater than about 30 wt %, or greater than about 40 wt %. In some embodiments, the percentage of cholesterol-based lipid in the lipid nanoparticle may be no more than about 5 wt %, no more than about 10 wt %, no more than about 20 wt %, no more than about 30 wt %, or no more than about 40 wt %.

PEG-Modified Lipids

In some embodiments, the liposome comprises one or more PEGylated lipids.

For example, the use of polyethylene glycol (PEG)-modified phospholipids and derivatized lipids such as derivatized ceramides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle).

Contemplated PEG-modified lipids include, but are not limited to, a polyethylene glycol chain of up to 5 kDa in length covalently attached to a lipid with alkyl chain(s) of C₆-C₂₀ length. In some embodiments, a PEG-modified or PEGylated lipid is PEGylated cholesterol or PEG-2K. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C₁₄ or C₁₈).

The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle. In some embodiments, one or more PEG-modified lipids constitute about 4% of the total lipids by molar ratio. In some embodiments, one or more PEG-modified lipids constitute about 5% of the total lipids by molar ratio. In some embodiments, one or more PEG-modified lipids constitute about 6% of the total lipids by molar ratio.

Amphiphilic Block Copolymers

In some embodiments, a suitable delivery vehicle contains amphiphilic block copolymers (e.g., poloxamers).

Various amphiphilic block copolymers may be used to practice the present invention. In some embodiments, an amphiphilic block copolymer is also referred to as a surfactant or a non-ionic surfactant.

In some embodiments, an amphiphilic polymer suitable for the invention is selected from poloxamers (Pluronic®), poloxamines (Tetronic®), polyoxyethylene glycol sorbitan alkyl esters (polysorbates) and polyvinyl pyrrolidones (PVPs).

Poloxamers

In some embodiments, a suitable amphiphilic polymer is a poloxamer. For example, a suitable poloxamer is of the following structure:

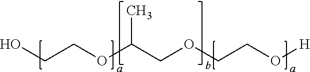

wherein a is an integer between 10 and 150 and b is an integer between 20 and 60. For example, a is about 12 and b is about 20, or a is about 80 and b is about 27, or a is about 64 and b is about 37, or a is about 141 and b is about 44, or a is about 101 and b is about 56.

In some embodiments, a poloxamer suitable for the invention has ethylene oxide units from about 10 to about 150. In some embodiments, a poloxamer has ethylene oxide units from about 10 to about 100.

In some embodiments, a suitable poloxamer is poloxamer 84. In some embodiments, a suitable poloxamer is poloxamer 101. In some embodiments, a suitable poloxamer is poloxamer 105. In some embodiments, a suitable poloxamer is poloxamer 108. In some embodiments, a suitable poloxamer is poloxamer 122. In some embodiments, t a suitable poloxamer is poloxamer 123. In some embodiments, a suitable poloxamer is poloxamer 124. In some embodiments, a suitable poloxamer is poloxamer 181. In some embodiments, a suitable poloxamer is poloxamer 182. In some embodiments, a suitable poloxamer is poloxamer 183. In some embodiments, a suitable poloxamer is poloxamer 184. In some embodiments, a suitable poloxamer is poloxamer 185. In some embodiments, a suitable poloxamer is poloxamer 188. In some embodiments, a suitable poloxamer is poloxamer 212. In some embodiments, a suitable poloxamer is poloxamer 215. In some embodiments, a suitable poloxamer is poloxamer 217. In some embodiments, a suitable poloxamer is poloxamer 231. In some embodiments, a suitable poloxamer is poloxamer 234. In some embodiments, a suitable poloxamer is poloxamer 235. In some embodiments, a suitable poloxamer is poloxamer 237. In some embodiments, a suitable poloxamer is poloxamer 238. In some embodiments, a suitable poloxamer is poloxamer 282. In some embodiments, a suitable poloxamer is poloxamer 284. In some embodiments, a suitable poloxamer is poloxamer 288. In some embodiments, a suitable poloxamer is poloxamer 304. In some embodiments, a suitable poloxamer is poloxamer 331. In some embodiments, a suitable poloxamer is poloxamer 333. In some embodiments, a suitable poloxamer is poloxamer 334. In some embodiments, a suitable poloxamer is poloxamer 335. In some embodiments, a suitable poloxamer is poloxamer 338. In some embodiments, a suitable poloxamer is poloxamer 401. In some embodiments, a suitable poloxamer is poloxamer 402. In some embodiments, a suitable poloxamer is poloxamer 403. In some embodiments, a suitable poloxamer is poloxamer 407. In some embodiments, a suitable poloxamer is a combination thereof.

In some embodiments, a suitable poloxamer has an average molecular weight of about 4,000 g/mol to about 20,000 g/mol. In some embodiments, a suitable poloxamer has an average molecular weight of about 1,000 g/mol to about 50,000 g/mol. In some embodiments, a suitable poloxamer has an average molecular weight of about 1,000 g/mol. In some embodiments, a suitable poloxamer has an average molecular weight of about 2,000 g/mol. In some embodiments, a suitable poloxamer has an average molecular weight of about 3,000 g/mol. In some embodiments, a suitable poloxamer has an average molecular weight of about 4,000 g/mol. In some embodiments, a suitable poloxamer has an average molecular weight of about 5,000 g/mol. In some embodiments, a suitable poloxamer has an average molecular weight of about 6,000 g/mol. In some embodiments, a suitable poloxamer has an average molecular weight of about 7,000 g/mol. In some embodiments, a suitable poloxamer has an average molecular weight of about 8,000 g/mol. In some embodiments, a suitable poloxamer has an average molecular weight of about 9,000 g/mol. In some embodiments, a suitable poloxamer has an average molecular weight of about 10,000 g/mol. In some embodiments, a suitable poloxamer has an average molecular weight of about 20,000 g/mol. In some embodiments, a suitable poloxamer has an average molecular weight of about 25,000 g/mol. In some embodiments, a suitable poloxamer has an average molecular weight of about 30,000 g/mol. In some embodiments, a suitable poloxamer has an average molecular weight of about 40,000 g/mol. In some embodiments, a suitable poloxamer has an average molecular weight of about 50,000 g/mol.

Other Amphiphilic Polymers

In some embodiments, an amphiphilic polymer is a poloxamine, e.g., tetronic 304 or tetronic 904.

In some embodiments, an amphiphilic polymer is a polyvinylpyrrolidone (PVP), such as PVP with molecular weight of 3 kDa, 10 kDa, or 29 kDa.

In some embodiments, an amphiphilic polymer is a polyethylene glycol ether (Brij), polysorbate, sorbitan, and derivatives thereof. In some embodiments, an amphiphilic polymer is a polysorbate, such as PS 20.

In some embodiments, an amphiphilic polymer is polyethylene glycol ether (Brij), poloxamer, polysorbate, sorbitan, or derivatives thereof.

In some embodiments, an amphiphilic polymer is a polyethylene glycol ether. In some embodiments, a suitable polyethylene glycol ether is a compound of Formula (S-1):

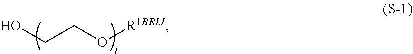

or a salt or isomer thereof, wherein:

t is an integer between 1 and 100;

R^{1BRU} independently is C₁₀₋₄₀ alkyl, C₁₀₋₄₀ alkenyl, or C₁₀₋₄₀ alkynyl; and optionally one or more methylene groups of R^{5PEG} are independently replaced with C₃₋₁₀ carbocyclylene, 4 to 10 membered heterocyclylene, C₆₋₁₀ arylene, 4 to 10 membered heteroarylene, —N(R^{N})—, —O—, —S—, —C(O)—, —C(O)N(R^{N})—, —NR^{N}C(O)—, —NRC(O)N(R)—, —C(O)O—, —OC(O)—, —OC(O)O—, —OC(O)N(R^{N})—, —NR^{N}C(O)O—, —C(O)S—, —SC(O)—, —C(═NR^{N})—, —C(═NR)N(R)—, —NRNC(═NR^{N})—, —NR^{N}C(═NR^{N})N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N})S(O)—, —S(O)N(R^{N})—, —N(R^{N})S(O)N(R^{N})—, —OS(O)N(R^{N})—, —N(R^{N})S(O)O—, —S(O)₂—, —N(R^{N})S(O)₂—, —S(O)₂N(R^{N})—, —N(R^{N})S(O)₂N(R^{N})—, —OS(O)₂N(R^{N})—, or —N(R^{N})S(O)₂O—; and each instance of R^{N} is independently hydrogen, C₁₋₆ alkyl, or a nitrogen protecting group.

In some embodiment, R^{1BRU} is C is alkyl. For example, the polyethylene glycol ether is a compound of Formula (S-1a):

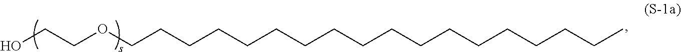

or a salt or isomer thereof, wherein s is an integer between 1 and 100.

In some embodiments, R^{1BRU} is C is alkenyl. For example, a suitable polyethylene glycol ether is a compound of Formula (S-1b):

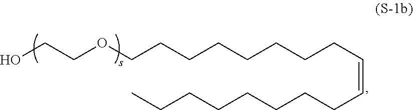

or a salt or isomer thereof, wherein s is an integer between 1 and 100.

Typically, an amphiphilic polymer (e.g., a poloxamer) is present in a formulation at an amount lower than its critical micelle concentration (CMC). In some embodiments, an amphiphilic polymer (e.g., a poloxamer) is present in the mixture at an amount about 1%, about 2%, about 3%, about 4%, about 5%, about 6%, about 7%, about 8%, about 9%, about 10%, about 15%, about 20%, about 25%, about 30%, about 35%, about 40%, about 45%, or about 50% lower than its CMC. In some embodiments, an amphiphilic polymer (e.g., a poloxamer) is present in the mixture at an amount about 0.9%, 0.8%, 0.7%, 0.6%, 0.5%, 0.4%, 0.3%, 0.2%, 0.1% lower than its CMC. In some embodiments, an amphiphilic polymer (e.g., a poloxamer) is present in the mixture at an amount about 55%, 60%, 65%, 70%, 75%, 80%, 90%, or 95% lower than its CMC.

In some embodiments, less than about 0.1%, 0.09%, 0.08%, 0.07%, 0.06%, 0.05%, 0.04%, 0.03%, 0.02%, or 0.01% of the original amount of the amphiphilic polymer (e.g., the poloxamer) present in the formulation remains upon removal. In some embodiments, a residual amount of the amphiphilic polymer (e.g., the poloxamer) remains in a formulation upon removal. As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

In some embodiments, a suitable delivery vehicle comprises less than 5% amphiphilic block copolymers (e.g., poloxamers). In some embodiments, a suitable delivery vehicle comprises less than 3% amphiphilic block copolymers (e.g., poloxamers). In some embodiments, a suitable delivery vehicle comprises less than 2.5% amphiphilic block copolymers (e.g., poloxamers). In some embodiments, suitable delivery vehicle comprises less than 2% amphiphilic block copolymers (e.g., poloxamers). In some embodiments, a suitable delivery vehicle comprises less than 1.5% amphiphilic block copolymers (e.g., poloxamers). In some embodiments, a suitable delivery vehicle comprises less than 1% amphiphilic block copolymers (e.g., poloxamers). In some embodiments, a suitable delivery vehicle comprises less than 0.5% (e.g., less than 0.4%, 0.3%, 0.2%, 0.1%) amphiphilic block copolymers (e.g., poloxamers). In some embodiments, a suitable delivery vehicle comprises less than 0.09%, 0.08%, 0.07%, 0.06%, 0.05%, 0.04%, 0.03%, 0.02%, or 0.01% amphiphilic block copolymers (e.g., poloxamers). In some embodiments, a suitable delivery vehicle comprises less than 0.01% amphiphilic block copolymers (e.g., poloxamers). In some embodiments, a suitable delivery vehicle contains a residual amount of amphiphilic polymers (e.g., poloxamers). As used herein, a residual amount means a remaining amount after substantially all of the substance (an amphiphilic polymer described herein such as a poloxamer) in a composition is removed. A residual amount may be detectable using a known technique qualitatively or quantitatively. A residual amount may not be detectable using a known technique.

Polymers

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

According to various embodiments, the selection of cationic lipids, non-cationic lipids, PEG-modified lipids, cholesterol-based lipids, and/or amphiphilic block copolymers which comprise the lipid nanoparticle, as well as the relative molar ratio of such components (lipids) to each other, is based upon the characteristics of the selected lipid(s), the nature of the intended target cells, the characteristics of the nucleic acid to be delivered. Additional considerations include, for example, the saturation of the alkyl chain, as well as the size, charge, pH, pKa, fusogenicity and toxicity of the selected lipid(s). Thus the molar ratios may be adjusted accordingly.

Ratio of Distinct Lipid Components

A suitable liposome for the present invention may include one or more of any of the cationic lipids, non-cationic lipids, cholesterol lipids, PEG-modified lipids, amphiphilic block copolymers and/or polymers described herein at various ratios. In some embodiments, a lipid nanoparticle comprises five and no more than five distinct components of nanoparticle. In some embodiments, a lipid nanoparticle comprises four and no more than four distinct components of nanoparticle. In some embodiments, a lipid nanoparticle comprises three and no more than three distinct components of nanoparticle. As non-limiting examples, a suitable liposome formulation may include a combination selected from cKK-E12, DOPE, cholesterol and DMG-PEG2K; C₁₂-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C₁₂₋₂₀₀, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C₁₂-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5.

In embodiments where a lipid nanoparticle comprises three and no more than three distinct components of lipids, the ratio of total lipid content (i.e., the ratio of lipid component (1):lipid component (2):lipid component (3)) can be represented as x:y:z, wherein

(y+z)=100−x.

In some embodiments, each of “x,” “y,” and “z” represents molar percentages of the three distinct components of lipids, and the ratio is a molar ratio.

In some embodiments, each of “x,” “y,” and “z” represents weight percentages of the three distinct components of lipids, and the ratio is a weight ratio.

In some embodiments, lipid component (1), represented by variable “x,” is a sterol-based cationic lipid.

In some embodiments, lipid component (2), represented by variable “y,” is a helper lipid.

In some embodiments, lipid component (3), represented by variable “z” is a PEG lipid.

In some embodiments, variable “x,” representing the molar percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the molar percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the molar percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “x,” representing the weight percentage of lipid component (1) (e.g., a sterol-based cationic lipid), is at least about 10%, about 20%, about 30%, about 40%, about 50%, about 55%, about 60%, about 65%, about 70%, about 75%, about 80%, about 85%, about 90%, or about 95%.

In some embodiments, variable “x,” representing the weight percentage of lipid component (1) (e.g., a sterol-based cationic lipid), is no more than about 95%, about 90%, about 85%, about 80%, about 75%, about 70%, about 65%, about 60%, about 55%, about 50%, about 40%, about 30%, about 20%, or about 10%. In embodiments, variable “x” is no more than about 65%, about 60%, about 55%, about 50%, about 40%.

In some embodiments, variable “x,” representing the weight percentage of lipid component (1) (e.g., a sterol-based cationic lipid), is: at least about 50% but less than about 95%; at least about 50% but less than about 90%; at least about 50% but less than about 85%; at least about 50% but less than about 80%; at least about 50% but less than about 75%; at least about 50% but less than about 70%; at least about 50% but less than about 65%; or at least about 50% but less than about 60%. In embodiments, variable “x” is at least about 50% but less than about 70%; at least about 50% but less than about 65%; or at least about 50% but less than about 60%.

In some embodiments, variable “z,” representing the molar percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the molar percentage of lipid component (3) (e.g., a PEG lipid) is about 1%, 2%, 3%, 4%, 5%, 6%, 7%, 8%, 9%, 10%. In embodiments, variable “z,” representing the molar percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

In some embodiments, variable “z,” representing the weight percentage of lipid component (3) (e.g., a PEG lipid) is no more than about 1%, 2%, 3%, 4%, 5%, 6%, 7%, 8%, 9%, 10%, 15%, 20%, or 25%. In embodiments, variable “z,” representing the weight percentage of lipid component (3) (e.g., a PEG lipid) is about 1%, 2%, 3%, 4%, 5%, 6%, 7%, 8%, 9%, 10%. In embodiments, variable “z,” representing the weight percentage of lipid component (3) (e.g., a PEG lipid) is about 1% to about 10%, about 2% to about 10%, about 3% to about 10%, about 4% to about 10%, about 1% to about 7.5%, about 2.5% to about 10%, about 2.5% to about 7.5%, about 2.5% to about 5%, about 5% to about 7.5%, or about 5% to about 10%.

For compositions having three and only three distinct lipid components, variables “x,” “y,” and “z” may be in any combination so long as the total of the three variables sums to 100% of the total lipid content.

Formation of Liposomes Encapsulating mRNA

The liposomal transfer vehicles for use in the compositions of the invention can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase may then be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

Various methods are described in published U.S. Application No. US 2011/0244026, published U.S. Application No. US 2016/0038432, published U.S. Application No. US 2018/0153822, published U.S. Application No. US 2018/0125989 and U.S. Provisional Application No. 62/877,597, filed Jul. 23, 2019 and can be used to practice the present invention, all of which are incorporated herein by reference. As used herein, Process A refers to a conventional method of encapsulating mRNA by mixing mRNA with a mixture of lipids, without first pre-forming the lipids into lipid nanoparticles, as described in US 2016/0038432. As used herein, Process B refers to a process of encapsulating messenger RNA (mRNA) by mixing pre-formed lipid nanoparticles with mRNA, as described in US 2018/0153822.

Briefly, the process of preparing mRNA- or MCNA-loaded lipid liposomes includes a step of heating one or more of the solutions (i.e., applying heat from a heat source to the solution) to a temperature (or to maintain at a temperature) greater than ambient temperature, the one more solutions being the solution comprising the pre-formed lipid nanoparticles, the solution comprising the mRNA and the mixed solution comprising the lipid nanoparticle encapsulated mRNA. In some embodiments, the process includes the step of heating one or both of the mRNA solution and the pre-formed lipid nanoparticle solution, prior to the mixing step. In some embodiments, the process includes heating one or more one or more of the solution comprising the pre-formed lipid nanoparticles, the solution comprising the mRNA and the solution comprising the lipid nanoparticle encapsulated mRNA, during the mixing step. In some embodiments, the process includes the step of heating the lipid nanoparticle encapsulated mRNA, after the mixing step. In some embodiments, the temperature to which one or more of the solutions is heated (or at which one or more of the solutions is maintained) is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature greater than ambient temperature to which one or more of the solutions is heated is about 65° C.

Various methods may be used to prepare an mRNA solution suitable for the present invention. In some embodiments, mRNA may be directly dissolved in a buffer solution described herein. In some embodiments, an mRNA solution may be generated by mixing an mRNA stock solution with a buffer solution prior to mixing with a lipid solution for encapsulation. In some embodiments, an mRNA solution may be generated by mixing an mRNA stock solution with a buffer solution immediately before mixing with a lipid solution for encapsulation. In some embodiments, a suitable mRNA stock solution may contain mRNA in water at a concentration at or greater than about 0.2 mg/ml, 0.4 mg/ml, 0.5 mg/ml, 0.6 mg/ml, 0.8 mg/ml, 1.0 mg/ml, 1.2 mg/ml, 1.4 mg/ml, 1.5 mg/ml, or 1.6 mg/ml, 2.0 mg/ml, 2.5 mg/ml, 3.0 mg/ml, 3.5 mg/ml, 4.0 mg/ml, 4.5 mg/ml, or 5.0 mg/ml.

In some embodiments, an mRNA stock solution is mixed with a buffer solution using a pump. Exemplary pumps include but are not limited to gear pumps, peristaltic pumps and centrifugal pumps.

Typically, the buffer solution is mixed at a rate greater than that of the mRNA stock solution. For example, the buffer solution may be mixed at a rate at least 1×, 2×, 3×, 4×, 5×, 6×, 7×, 8×, 9×, 10×, 15×, or 20× greater than the rate of the mRNA stock solution. In some embodiments, a buffer solution is mixed at a flow rate ranging between about 100-6000 ml/minute (e.g., about 100-300 ml/minute, 300-600 ml/minute, 600-1200 ml/minute, 1200-2400 ml/minute, 2400-3600 ml/minute, 3600-4800 ml/minute, 4800-6000 ml/minute, or 60-420 ml/minute). In some embodiments, a buffer solution is mixed at a flow rate of or greater than about 60 ml/minute, 100 ml/minute, 140 ml/minute, 180 ml/minute, 220 ml/minute, 260 ml/minute, 300 ml/minute, 340 ml/minute, 380 ml/minute, 420 ml/minute, 480 ml/minute, 540 ml/minute, 600 ml/minute, 1200 ml/minute, 2400 ml/minute, 3600 ml/minute, 4800 ml/minute, or 6000 ml/minute.

In some embodiments, an mRNA stock solution is mixed at a flow rate ranging between about 10-600 ml/minute (e.g., about 5-50 ml/minute, about 10-30 ml/minute, about 30-60 ml/minute, about 60-120 ml/minute, about 120-240 ml/minute, about 240-360 ml/minute, about 360-480 ml/minute, or about 480-600 ml/minute). In some embodiments, an mRNA stock solution is mixed at a flow rate of or greater than about 5 ml/minute, 10 ml/minute, 15 ml/minute, 20 ml/minute, 25 ml/minute, 30 ml/minute, 35 ml/minute, 40 ml/minute, 45 ml/minute, 50 ml/minute, 60 ml/minute, 80 ml/minute, 100 ml/minute, 200 ml/minute, 300 ml/minute, 400 ml/minute, 500 ml/minute, or 600 ml/minute.

According to the present invention, a lipid solution contains a mixture of lipids suitable to form lipid nanoparticles for encapsulation of mRNA. In some embodiments, a suitable lipid solution is ethanol based. For example, a suitable lipid solution may contain a mixture of desired lipids dissolved in pure ethanol (i.e., 100% ethanol). In another embodiment, a suitable lipid solution is isopropyl alcohol based. In another embodiment, a suitable lipid solution is dimethylsulfoxide-based. In another embodiment, a suitable lipid solution is a mixture of suitable solvents including, but not limited to, ethanol, isopropyl alcohol and dimethylsulfoxide.

A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.5-90 mg/ml, 1.0-80 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, or 10 mg/ml.

Any desired lipids may be mixed at any ratios suitable for encapsulating mRNAs. In some embodiments, a suitable lipid solution contains a mixture of desired lipids including cationic lipids, helper lipids (e.g. non cationic lipids and/or cholesterol lipids), amphiphilic block copolymers (e.g. poloxamers) and/or PEGylated lipids. In some embodiments, a suitable lipid solution contains a mixture of desired lipids including one or more cationic lipids, one or more helper lipids (e.g. non cationic lipids and/or cholesterol lipids) and one or more PEGylated lipids.

In certain embodiments, provided compositions comprise a liposome wherein the mRNA is associated on both the surface of the liposome and encapsulated within the same liposome. For example, during preparation of the compositions of the present invention, cationic liposomes may associate with the mRNA or MCNA through electrostatic interactions.

In some embodiments, the compositions and methods of the invention comprise mRNA encapsulated in a liposome. In some embodiments, the one or more mRNA species may be encapsulated in the same liposome. In some embodiments, the one or more mRNA species may be encapsulated in different liposomes. In some embodiments, the mRNA is encapsulated in one or more liposomes, which differ in their lipid composition, molar ratio of lipid components, size, charge (zeta potential), targeting ligands and/or combinations thereof. In some embodiments, the one or more liposome may have a different composition of sterol-based cationic lipids, neutral lipid, PEG-modified lipid and/or combinations thereof. In some embodiments the one or more liposomes may have a different molar ratio of cholesterol-based cationic lipid, neutral lipid, and PEG-modified lipid used to create the liposome.

The process of incorporation of a desired nucleic acid (e.g., mRNA or MCNA) into a liposome is often referred to as “loading”. Exemplary methods are described in Lasic, et al. FEBS Lett., 312: 255-258, 1992, which is incorporated herein by reference. The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating an mRNA into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in some embodiments, a suitable delivery vehicle is capable of enhancing the stability of the mRNA contained therein and/or facilitate the delivery of therapeutic agent (e.g., mRNA or MCNA) to the target cell or tissue.

Suitable liposomes in accordance with the present invention may be made in various sizes. In some embodiments, provided liposomes may be made smaller than previously known liposomes. In some embodiments, decreased size of liposomes is associated with more efficient delivery of therapeutic agent (e.g., mRNA or MCNA). Selection of an appropriate liposome size may take into consideration the site of the target cell or tissue and to some extent the application for which the liposome is being made.

In some embodiments, an appropriate size of liposome is selected to facilitate systemic distribution of antibody encoded by the mRNA. In some embodiments, it may be desirable to limit transfection of the mRNA to certain cells or tissues. For example, to target hepatocytes a liposome may be sized such that its dimensions are smaller than the fenestrations of the endothelial layer lining hepatic sinusoids in the liver; in such cases the liposome could readily penetrate such endothelial fenestrations to reach the target hepatocytes.

Alternatively or additionally, a liposome may be sized such that the dimensions of the liposome are of a sufficient diameter to limit or expressly avoid distribution into certain cells or tissues.

A variety of alternative methods known in the art are available for sizing of a population of liposomes. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomes may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

Provided Nanoparticles Encapsulating mRNA

In some embodiments, majority of purified nanoparticles in a composition, i.e., greater than about 50%, 55%, 60%, 65%, 70%, 75%, 80%, 85%, 90%, 95%, 96%, 97%, 98%, or 99% of the nanoparticles, have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm). In some embodiments, substantially all of the purified nanoparticles have a size of about 150 nm (e.g., about 145 nm, about 140 nm, about 135 nm, about 130 nm, about 125 nm, about 120 nm, about 115 nm, about 110 nm, about 105 nm, about 100 nm, about 95 nm, about 90 nm, about 85 nm, or about 80 nm).

In some embodiments, a lipid nanoparticle has an average size of less than 150 nm. In some embodiments, a lipid nanoparticle has an average size of less than 120 nm. In some embodiments, a lipid nanoparticle has an average size of less than 100 nm. In some embodiments, a lipid nanoparticle has an average size of less than 90 nm. In some embodiments, a lipid nanoparticle has an average size of less than 80 nm. In some embodiments, a lipid nanoparticle has an average size of less than 70 nm. In some embodiments, a lipid nanoparticle has an average size of less than 60 nm. In some embodiments, a lipid nanoparticle has an average size of less than 50 nm. In some embodiments, a lipid nanoparticle has an average size of less than 30 nm. In some embodiments, a lipid nanoparticle has an average size of less than 20 nm.

In some embodiments, the dispersity, or measure of heterogeneity in size of molecules (PDI), of nanoparticles in a composition provided by the present invention is less than about 0.5. In some embodiments, a lipid nanoparticle has a PDI of less than about 0.5. In some embodiments, a lipid nanoparticle has a PDI of less than about 0.4. In some embodiments, a lipid nanoparticle has a PDI of less than about 0.3. In some embodiments, a lipid nanoparticle has a PDI of less than about 0.28. In some embodiments, a lipid nanoparticle has a PDI of less than about 0.25. In some embodiments, a lipid nanoparticle has a PDI of less than about 0.23. In some embodiments, a lipid nanoparticle has a PDI of less than about 0.20. In some embodiments, a lipid nanoparticle has a PDI of less than about 0.18. In some embodiments, a lipid nanoparticle has a PDI of less than about 0.16. In some embodiments, a lipid nanoparticle has a PDI of less than about 0.14. In some embodiments, a lipid nanoparticle has a PDI of less than about 0.12. In some embodiments, a lipid nanoparticle has a PDI of less than about 0.10. In some embodiments, a lipid nanoparticle has a PDI of less than about 0.08.

In some embodiments, greater than about 75%, 80%, 85%, 90%, 95%, 96%, 97%, 98%, or 99% of the purified lipid nanoparticles in a composition provided by the present invention encapsulate an mRNA within each individual particle. In some embodiments, substantially all of the purified lipid nanoparticles in a composition encapsulate an mRNA within each individual particle. In some embodiments, a lipid nanoparticle has an encapsulation efficiency of between 50% and 99%. In some embodiments, a lipid nanoparticle has an encapsulation efficiency of greater than about 60%. In some embodiments, a lipid nanoparticle has an encapsulation efficiency of greater than about 65%. In some embodiments, a lipid nanoparticle has an encapsulation efficiency of greater than about 70%. In some embodiments, a lipid nanoparticle has an encapsulation efficiency of greater than about 75%. In some embodiments, a lipid nanoparticle has an encapsulation efficiency of greater than about 80%. In some embodiments, a lipid nanoparticle has an encapsulation efficiency of greater than about 85%. In some embodiments, a lipid nanoparticle has an encapsulation efficiency of greater than about 90%. In some embodiments, a lipid nanoparticle has an encapsulation efficiency of greater than about 92%. In some embodiments, a lipid nanoparticle has an encapsulation efficiency of greater than about 95%. In some embodiments, a lipid nanoparticle has an encapsulation efficiency of greater than about 98%. In some embodiments, a lipid nanoparticle has an encapsulation efficiency of greater than about 99%.

In some embodiments, a lipid nanoparticle has a N/P ratio of between 1 and 10. As used herein, the term “N/P ratio” refers to a molar ratio of positively charged molecular units in the cationic lipids in a lipid nanoparticle relative to negatively charged molecular units in the mRNA encapsulated within that lipid nanoparticle. As such, N/P ratio is typically calculated as the ratio of moles of amine groups in cationic lipids in a lipid nanoparticle relative to moles of phosphate groups in mRNA encapsulated within that lipid nanoparticle. In some embodiments, a lipid nanoparticle has a N/P ratio above 1. In some embodiments, a lipid nanoparticle has a N/P ratio of about 1. In some embodiments, a lipid nanoparticle has a N/P ratio of about 2. In some embodiments, a lipid nanoparticle has a N/P ratio of about 3. In some embodiments, a lipid nanoparticle has a N/P ratio of about 4. In some embodiments, a lipid nanoparticle has a N/P ratio of about 5. In some embodiments, a lipid nanoparticle has a N/P ratio of about 6. In some embodiments, a lipid nanoparticle has a N/P ratio of about 7. In some embodiments, a lipid nanoparticle has a N/P ratio of about 8.

In some embodiments, a composition according to the present invention contains at least about 0.5 mg, 1 mg, 5 mg, 10 mg, 100 mg, 500 mg, or 1000 mg of encapsulated mRNA. In some embodiments, a composition contains about 0.1 mg to 1000 mg of encapsulated mRNA. In some embodiments, a composition contains at least about 0.5 mg of encapsulated mRNA. In some embodiments, a composition contains at least about 0.8 mg of encapsulated mRNA. In some embodiments, a composition contains at least about 1 mg of encapsulated mRNA. In some embodiments, a composition contains at least about 5 mg of encapsulated mRNA. In some embodiments, a composition contains at least about 8 mg of encapsulated mRNA. In some embodiments, a composition contains at least about 10 mg of encapsulated mRNA. In some embodiments, a composition contains at least about 50 mg of encapsulated mRNA. In some embodiments, a composition contains at least about 100 mg of encapsulated mRNA. In some embodiments, a composition contains at least about 500 mg of encapsulated mRNA. In some embodiments, a composition contains at least about 1000 mg of encapsulated mRNA.

### Therapeutic Use of Compositions

To facilitate expression of mRNA in vivo, delivery vehicles such as liposomes can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

In some embodiments, a composition comprises mRNA encapsulated or complexed with a delivery vehicle. In some embodiments, the delivery vehicle is selected from the group consisting of liposomes, lipid nanoparticles, solid-lipid nanoparticles, polymers, viruses, sol-gels, and nanogels.

Provided mRNA-loaded nanoparticles, and compositions containing the same, may be administered and dosed in accordance with current medical practice, taking into account the clinical condition of the subject, the site and method of administration, the scheduling of administration, the subject's age, sex, body weight and other factors relevant to clinicians of ordinary skill in the art. The “effective amount” for the purposes herein may be determined by such relevant considerations as are known to those of ordinary skill in experimental clinical research, pharmacological, clinical, and medical arts. In some embodiments, the amount administered is effective to achieve at least some stabilization, improvement or elimination of symptoms and other indicators as are selected as appropriate measures of disease progress, regression or improvement by those of skill in the art. For example, a suitable amount and dosing regimen is one that causes at least transient protein (e.g., enzyme) production.

The present invention provides methods of delivering mRNA for in vivo protein production, comprising administering mRNA to a subject in need of delivery. In some embodiments, mRNA is administered via a route of delivery selected from the group consisting of intravenous delivery, subcutaneous delivery, oral delivery, subdermal delivery, ocular delivery, intratracheal injection pulmonary delivery (e.g. nebulization), intramuscular delivery, intrathecal delivery, or intraarticular delivery.

Suitable routes of administration include, for example, oral, rectal, vaginal, transmucosal, pulmonary including intratracheal or inhaled, or intestinal administration; parenteral delivery, including intradermal, transdermal (topical), intramuscular, subcutaneous, intramedullary injections, as well as intrathecal, direct intraventricular, intravenous, intraperitoneal, or intranasal. In some embodiments, the intramuscular administration is to a muscle selected from the group consisting of skeletal muscle, smooth muscle and cardiac muscle. In some embodiments the administration results in delivery of the mRNA to a muscle cell. In some embodiments the administration results in delivery of the mRNA to a hepatocyte (i.e., liver cell). In a particular embodiment, the intramuscular administration results in delivery of the mRNA to a muscle cell.

Additional teaching of pulmonary delivery and nebulization are described in published U.S. Application No. US 2018/0125989 and published U.S. Application No. US 2018/0333457, each of which is incorporated by reference in its entirety.

Alternatively or additionally, mRNA-loaded nanoparticles and compositions of the invention may be administered in a local rather than systemic manner, for example, via injection of the pharmaceutical composition directly into a targeted tissue, preferably in a sustained release formulation. Local delivery can be affected in various ways, depending on the tissue to be targeted. For example, aerosols containing compositions of the present invention can be inhaled (for nasal, tracheal, or bronchial delivery); compositions of the present invention can be injected into the site of injury, disease manifestation, or pain, for example; compositions can be provided in lozenges for oral, tracheal, or esophageal application; can be supplied in liquid, tablet or capsule form for administration to the stomach or intestines, can be supplied in suppository form for rectal or vaginal application; or can even be delivered to the eye by use of creams, drops, or even injection. Formulations containing provided compositions complexed with therapeutic molecules or ligands can even be surgically administered, for example in association with a polymer or other structure or substance that can allow the compositions to diffuse from the site of implantation to surrounding cells. Alternatively, they can be applied surgically without the use of polymers or supports.

Provided methods of the present invention contemplate single as well as multiple administrations of a therapeutically effective amount of the therapeutic agents (e.g., mRNA) described herein. Therapeutic agents can be administered at regular intervals, depending on the nature, severity and extent of the subject's condition. In some embodiments, a therapeutically effective amount of the therapeutic agents (e.g., mRNA) of the present invention may be administered intrathecally periodically at regular intervals (e.g., once every year, once every six-months, once every five-months, once every three-months, bimonthly (once every two-months), monthly (once every month), biweekly (once every two-weeks), twice a month, once every 30-days, once every 28-days, once every 14-days, once every 10-days, once every 7-days, weekly, twice a week, daily, or continuously).

In some embodiments, provided liposomes and/or compositions are formulated such that they are suitable for extended-release of the mRNA contained therein. Such extended-release compositions may be conveniently administered to a subject at extended dosing intervals. For example, in one embodiment, the compositions of the present invention are administered to a subject twice a day, daily, or every other day. In a preferred embodiment, the compositions of the present invention are administered to a subject twice a week, once a week, once every 7-days, once every 10-days, once every 14-days, once every 28-days, once every 30-days, once every two-weeks, once every three-weeks, or more-preferably once every four-weeks, once-a-month, twice-a-month, once every six-weeks, once every eight-weeks, once every other month, once every three-months, once every four-months, once every six-months, once every eight-months, once every nine-months, or annually. Also contemplated are compositions and liposomes that are formulated for depot administration (e.g., intramuscularly, subcutaneously, intravitreally) to either deliver or release therapeutic agent (e.g., mRNA) over extended periods of time. Preferably, the extended-release means employed are combined with modifications made to the mRNA to enhance stability.

As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating a disease or disorder). For example, a therapeutically effective amount may be an amount sufficient to achieve a desired therapeutic and/or prophylactic effect. Generally, the amount of a therapeutic agent (e.g., mRNA) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges.

A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific protein employed; the duration of the treatment; and like factors as is well known in the medical arts.

In some embodiments, the therapeutically effective dose ranges from about 0.005 mg/kg body weight to 500 mg/kg body weight, e.g., from about 0.005 mg/kg body weight to 400 mg/kg body weight, from about 0.005 mg/kg body weight to 300 mg/kg body weight, from about 0.005 mg/kg body weight to 200 mg/kg body weight, from about 0.005 mg/kg body weight to 100 mg/kg body weight, from about 0.005 mg/kg body weight to 90 mg/kg body weight, from about 0.005 mg/kg body weight to 80 mg/kg body weight, from about 0.005 mg/kg body weight to 70 mg/kg body weight, from about 0.005 mg/kg body weight to 60 mg/kg body weight, from about 0.005 mg/kg body weight to 50 mg/kg body weight, from about 0.005 mg/kg body weight to 40 mg/kg body weight, from about 0.005 mg/kg body weight to 30 mg/kg body weight, from about 0.005 mg/kg body weight to 25 mg/kg body weight, from about 0.005 mg/kg body weight to 20 mg/kg body weight, from about 0.005 mg/kg body weight to 15 mg/kg body weight, from about 0.005 mg/kg body weight to 10 mg/kg body weight.

In some embodiments, the therapeutically effective dose is greater than about 0.1 mg/kg body weight, greater than about 0.5 mg/kg body weight, greater than about 1.0 mg/kg body weight, greater than about 3 mg/kg body weight, greater than about 5 mg/kg body weight, greater than about 10 mg/kg body weight, greater than about 15 mg/kg body weight, greater than about 20 mg/kg body weight, greater than about 30 mg/kg body weight, greater than about 40 mg/kg body weight, greater than about 50 mg/kg body weight, greater than about 60 mg/kg body weight, greater than about 70 mg/kg body weight, greater than about 80 mg/kg body weight, greater than about 90 mg/kg body weight, greater than about 100 mg/kg body weight, greater than about 150 mg/kg body weight, greater than about 200 mg/kg body weight, greater than about 250 mg/kg body weight, greater than about 300 mg/kg body weight, greater than about 350 mg/kg body weight, greater than about 400 mg/kg body weight, greater than about 450 mg/kg body weight, greater than about 500 mg/kg body weight. In a particular embodiment, the therapeutically effective dose is 1.0 mg/kg. In some embodiments, the therapeutically effective dose of 1.0 mg/kg is administered intramuscularly or intravenously.

Also contemplated herein are lyophilized pharmaceutical compositions comprising one or more of the liposomes disclosed herein and related methods for the use of such compositions as disclosed for example, in U.S. Provisional Application No. 61/494,882, filed Jun. 8, 2011, the teachings of which are incorporated herein by reference in their entirety. For example, lyophilized pharmaceutical compositions according to the invention may be reconstituted prior to administration or can be reconstituted in vivo. For example, a lyophilized pharmaceutical composition can be formulated in an appropriate dosage form (e.g., an intradermal dosage form such as a disk, rod or membrane) and administered such that the dosage form is rehydrated over time in vivo by the individual's bodily fluids.

Provided liposomes and compositions may be administered to any desired tissue. In some embodiments, the mRNA delivered by provided liposomes or compositions is expressed in the tissue in which the liposomes and/or compositions were administered. In some embodiments, the mRNA delivered is expressed in a tissue different from the tissue in which the liposomes and/or compositions were administered. Exemplary tissues in which delivered mRNA may be delivered and/or expressed include, but are not limited to the liver, kidney, heart, spleen, serum, brain, skeletal muscle, lymph nodes, skin, and/or cerebrospinal fluid.

In some embodiments, administering the provided composition results in an increased mRNA expression level in a biological sample from a subject as compared to a baseline expression level before treatment. Typically, the baseline level is measured immediately before treatment. Biological samples include, for example, whole blood, serum, plasma, urine and tissue samples (e.g., muscle, liver, skin fibroblasts). In some embodiments, administering the provided composition results in an increased mRNA expression level by at least about 10%, 20%, 30%, 40%, 50%, 60%, 70%, 80%, 90%, or 95% as compared to the baseline level immediately before treatment. In some embodiments, administering the provided composition results in an increased mRNA expression level as compared to an mRNA expression level in subjects who are not treated

According to various embodiments, the timing of expression of delivered mRNA can be tuned to suit a particular medical need. In some embodiments, the expression of the protein encoded by delivered mRNA is detectable 1, 2, 3, 6, 12, 24, 48, 72, and/or 96 hours after administration of provided liposomes and/or compositions. In some embodiments, the expression of the protein encoded by delivered mRNA is detectable one-week, two-weeks, and/or one-month after administration.

The present invention also provides delivering a composition having mRNA molecules encoding a peptide or polypeptide of interest for use in the treatment of a subject, e.g., a human subject or a cell of a human subject or a cell that is treated and delivered to a human subject.

### EXAMPLES

While certain compounds, compositions and methods of the present invention have been described with specificity in accordance with certain embodiments, the following examples serve only to illustrate the compounds of the invention and are not intended to limit the same.

### Example 1. Formulation of mRNA-LNP Composition for Rectal Delivery

This example illustrates an exemplary process of making a composition comprising mRNA encapsulated within lipid nanoparticles (LNPs) suitable for rectal delivery.

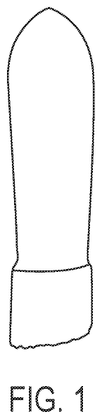

Messenger RNAs were encapsulated within lipid nanoparticles comprising ML-2: DOPE: Cholesterol: DMG-PEG (40:30:25:5) using Process B. As used herein, Process B refers to a process of encapsulating mRNA by mixing pre-formed lipid nanoparticles with mRNA, as described in US 2018/0153822, which is incorporated herein by its entirety. Suppositories were prepared at 10% w/v concentration of gelatin in LNPs. Briefly, gelatin was directly dissolved in LNPs at 65° C. within 5 minutes and poured into the disposable molds. The molds were then frozen at −80° C. The suppositories comprising mRNA encapsulated LNPs stayed intact. An exemplary suppository comprising mRNA-LNP is shown in FIG. 1.

Labrasol (permeability enhancer) solution was prepared by dissolving 153 mg of Labrasol in 1 mL of water.

Various amount of gelatin can be used to control the melting speed of the suppositories, hence controlling the release time of mRNA encapsulated within LNPs once administered to a subject. Additionally, viscosity modifying excipients can be added to control the release time.

### Example 2. Rectal Delivery of FFLuc mRNA-LNPs to Mice

This example illustrates successful rectal delivery of mRNA in lipid nanoparticles.

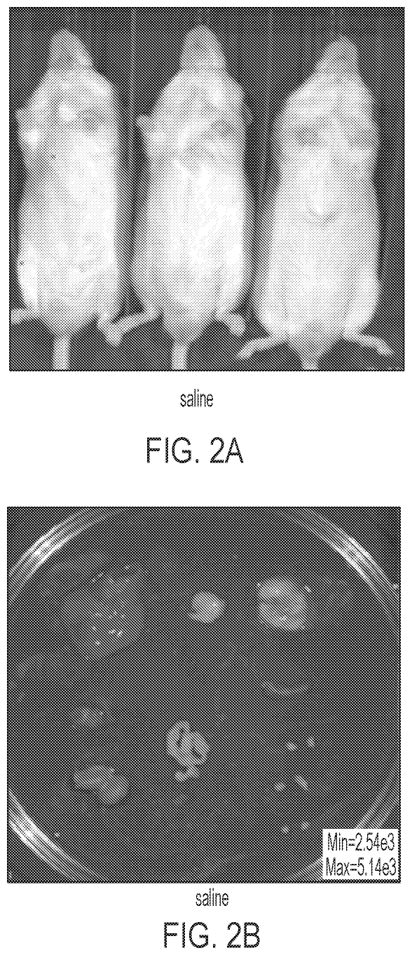

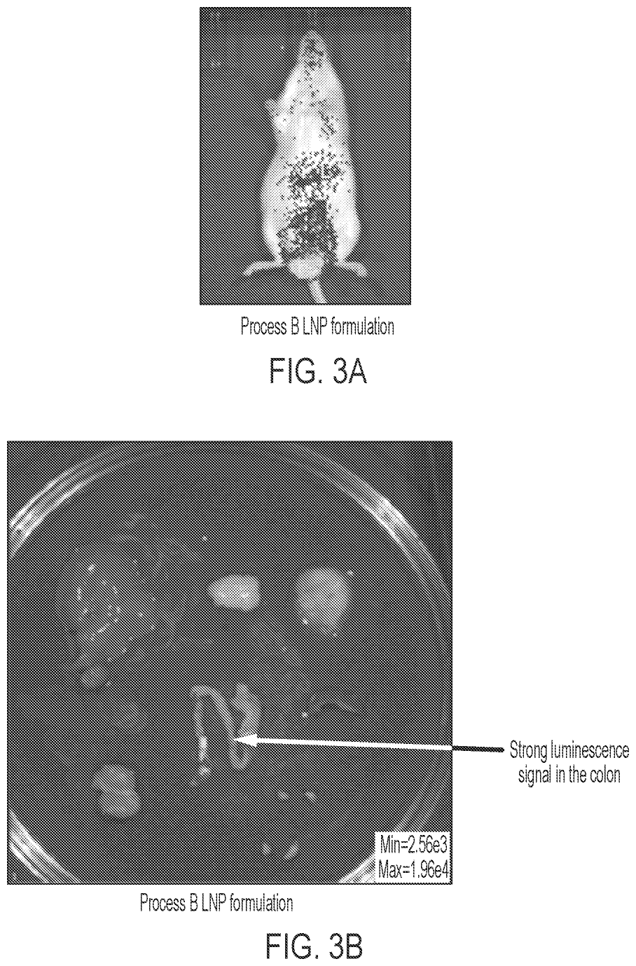

Firefly luciferase (FFLuc) mRNA encapsulated within lipid nanoparticles (0.2 mg per animal) or saline were dosed rectally in mice. Whole body and individual tissues were imaged after 24 hours of rectal administration, as shown in FIG. 2 and FIG. 3.

As exemplified in FIG. 2A and FIG. 2B, mice rectally dosed with saline did not show any luminescence, as expected. Mice rectally administered with FFLuc mRNA-LNPs exhibited luminescence, especially high luminescence at rectal area (FIG. 3A). Among the tissues, clones showed strong luminescence, as illustrated in FIG. 3B.

This example illustrates that mRNA-LNPs can successfully delivered rectally for in vivo expression of protein. The expression was detected rectum and colon.

### Example 3. Rectal Delivery of FFLuc mRNA-LNPs with Permeability Enhancer

This example illustrates successful delivery of mRNA in lipid nanoparticles with sodium caprate, a permeability enhancer. Rectal administration of mRNA-LNPs with sodium caprate significantly increased in vivo expression of protein.

One group of mice were administered with 0.2 mg of FFLuc mRNA-LNPs (Group 1) as described in Example 2. Second group of mice were pre-dosed with sodium caprate (200 mg/ml solution—50 ults injection) prior to rectal administration of 0.05 mg of FFLuc mRNA-LNPs (Group 2). Whole body and individual tissues were imaged after 24 hours of rectal administration.

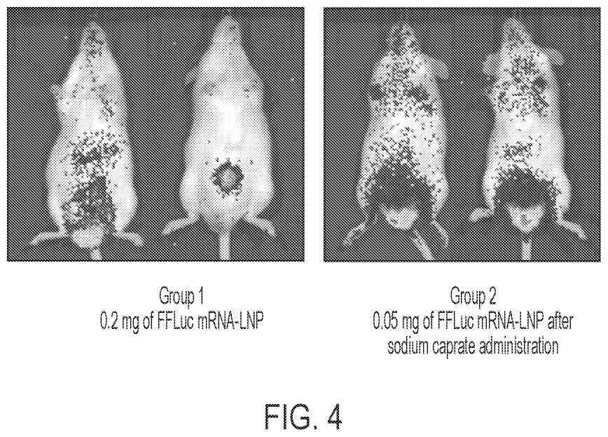

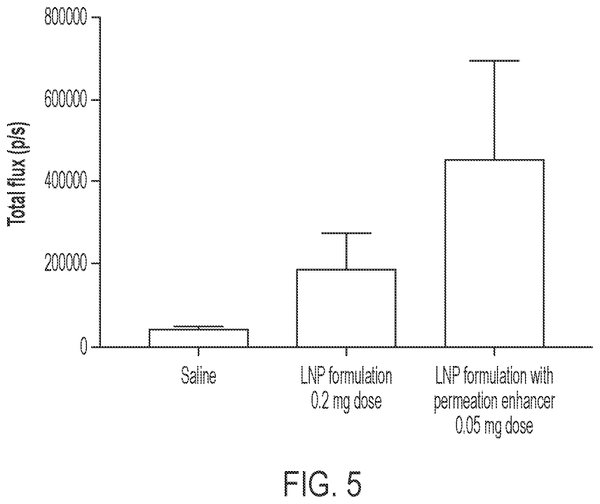

As shown in FIG. 4, mice rectally dosed with 0.05 mg of FFLuc mRNA-LNPs with sodium caprate showed significantly higher signal as compared to mice administered with 0.2 mg of FFLuc mRNA-LNPs. FIG. 5 shows that there was almost 2-fold increase in luminescence for Group 2, even though the dose of mRNA-LNP was 25% of the dose in Group 1.

This example illustrates that permeability enhancers can increase the expression of proteins delivered by mRNA-LNPs.

### Example 4. Rectal Delivery of FFLuc mRNA-LNPs in Suppositories to Mice and Rats

This example illustrates successful rectal delivery of mRNA-LNPs in suppository formulation. Rectal administration of mRNA-LNPs in suppositories significantly increased in vivo expression of protein, and protein expression was detected in various tissues.

Mice or rats were dosed rectally with 30 μl of Labrasol solution (153 mg/ml). After 30 minutes, suppository formulations of FFLuc mRNA-LNPs were administered rectally. Whole body and individual tissues were imaged after 24 hours of rectal administration.

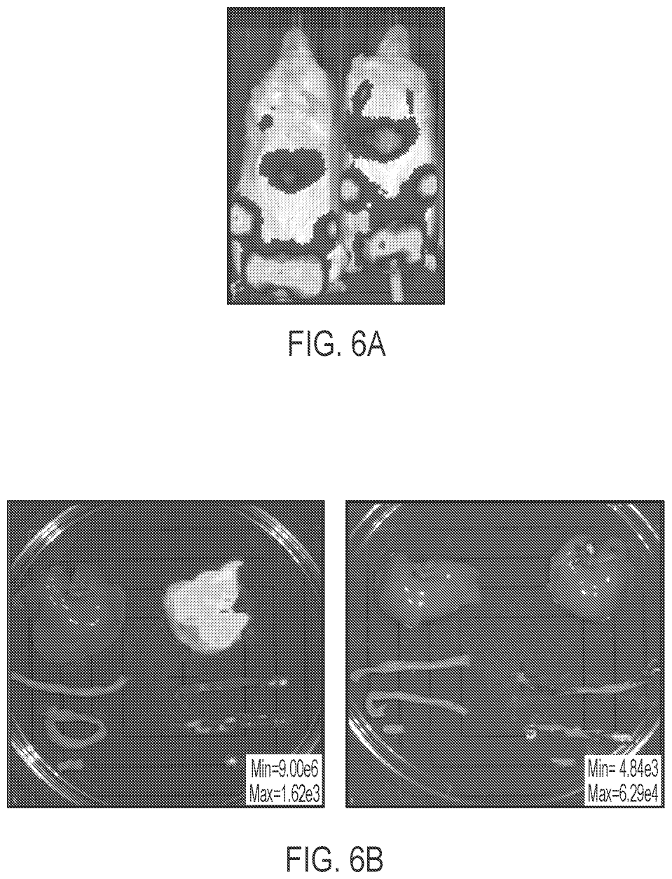

FIG. 6A shows that mice rectally administered with FFLuc mRNA-LNPs in suppositories showed significantly higher luminescence as compared to mice rectally administered with mRNA-LNPs without suppositories. Moreover, strong luminescence signal is observed in different tissues (e.g. rectum colon, liver, kidney, etc.). Two out of four rats showed luminescence in liver, colon and rectum, as exemplified in FIG. 6B. Less variability was observed in mice as compared to rats.

This example illustrates that when mRNA-LNPs are delivered in the form of suppository, a significant increase in protein expression is observed. The example also supports that suppositories can help mRNA-LNPs survive the RNase and mucus barrier, and also control the release of mRNA-LNPs inside the body of subjects once delivered. Moreover, in vivo protein expression was detected in various tissues including kidney. This is significant because targeted delivery of agents into mice kidney is known to be strenuous and may require laparotomy. Additionally, expression of FFL in liver suggests uptake of LNPs in the systemic circulation.

### Example 5. Rectal Delivery of EPO mRNA-LNPs in Suppositories to Rats

This example illustrates successful rectal delivery of mRNA-LNPs in suppositories for secreted proteins.

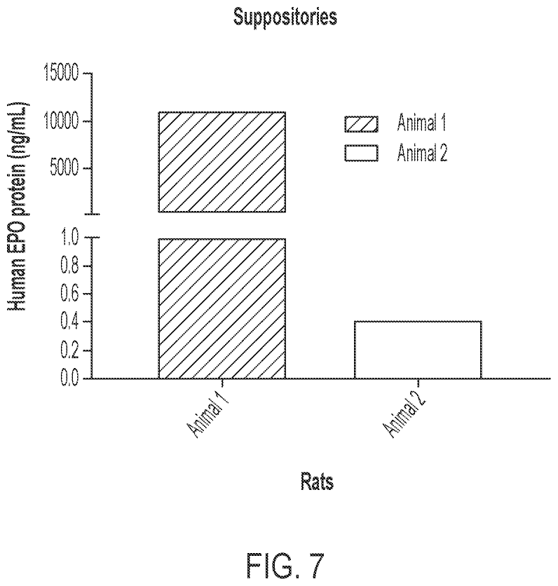

Rats were dosed rectally with 30 μl of Labrasol solution (153 mg/ml). After 30 minutes, suppository formulations of hEPO mRNA-LNPs were administered rectally. After 24 hours of administration, hEPO levels in serum was measured. As shown in FIG. 7, rats rectally dosed with hEPO mRNA-LNPs in suppositories showed detectable hEPO levels in serum. These levels are much higher than the normal physiological levels of EPO.

This example shows that rectal delivery of mRNA-LNPs in the form of suppository can successfully provide expressed protein in systemic circulation.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method for delivery of messenger RNA (mRNA) to a subject for in vivo production of a protein or a peptide in the subject, comprising administering to the subject by rectal delivery, a composition comprising an mRNA that encodes a protein or a peptide and is encapsulated within a lipid nanoparticle and wherein the administering of the composition results in expression of the protein or the peptide encoded by the mRNA that is detectable in the subject at least about 24 hours, about 48 hours, about 72 hours, or about 96 hours after administration.

2. The method of claim 1, wherein the protein or the peptide encoded by the mRNA is detectable in the subject's circulation at least about 24 hours, about 48 hours, about 72 hours, or about 96 hours after administration.

3. The method of claim 1, wherein the protein or the peptide encoded by the mRNA is detectable in the subject's liver at least about 24 hours, about 48 hours, about 72 hours, or about 96 hours after administration.

4. The method of claim 1, wherein the protein or the peptide encoded by the mRNA is detectable in the subject's kidney at least about 24 hours, about 48 hours, about 72 hours, or about 96 hours after administration.

5. The method of claim 1, wherein the protein or the peptide encoded by the mRNA is detectable in the subject's colon at least about 24 hours, about 48 hours, about 72 hours, or about 96 hours after administration.

6. The method of claim 1, wherein the protein or the peptide encoded by the mRNA is detectable in the subject's rectum at least about 24 hours, about 48 hours, about 72 hours, or about 96 hours after administration.

7. The method of claim 1, wherein the in vivo production of the protein or the peptide is in the subject's circulation, liver, kidney, colon and/or rectum.

8. The method of any one of the preceding claims, wherein the lipid nanoparticle comprises one or more cationic lipids, one or more non-cationic lipids and one or more PEG-modified lipids.

9. The method of any one of the preceding claims, wherein the lipid nanoparticle comprises cholesterol.

10. The method of any one of the preceding claims, wherein the rectal delivery is by suppository, enema, catheter or a bulb syringe.

11. The method of claim 10, wherein the rectal delivery is by suppository.

12. The method of claim 11, wherein the composition does not comprise a lipid-based suppository component.

13. The method of claim 12, wherein the lipid-based suppository component is cocoa butter, theobroma oil, synthetic fats or synthetic bases.

14. The method of any one of the preceding claims, wherein the composition comprises a permeability enhancer.

15. The method of claim 14, wherein the permeability enhancer is selected from bile salts, surfactants, fatty acids and derivatives, glycerides, chelators, salicylates, or polymers.

16. The method of claim 15, wherein the fatty acids and derivatives are selected from sorbitan laurate, sodium caprate, sucrose, palitate, lauroyl choline, sodium myristate, or palmitoyl carnitine.

17. The method of claim 14, wherein the permeability enhancer is a form of caprate.

18. The method of claim 18, wherein the caprate-based permeability enhancer is sodium caprate.

19. The method of claim 14, wherein the permeability enhancer is Labrasol®.

20. The method of any one of the preceding claims, wherein the composition comprises a water-based suppository component.

21. The method of claim 20, wherein the water-based suppository component is selected from glycerin, gelatin or polyethylene glycol (PEG), or combinations thereof.

22. The method of any one of the preceding claims, wherein the composition further comprises gelatin.

23. The method of claim 22, wherein the only water-based suppository component is gelatin.

24. The method of claim 23, wherein the composition comprises about 5% or more gelatin in water, 10% or more gelatin in water, 20% or more gelatin in water, 30% or more gelatin in water, or 50% or more gelatin in water.

25. The method of any one of the preceding claims, wherein the composition further comprises 0.25 mg/mL or greater mRNA, 0.5 mg/mL or greater mRNA, 0.75 mg/mL or greater mRNA, or 1 mg/mL or greater mRNA.

26. The method of claim 25, wherein the composition comprises 0.5 mg or greater mRNA, 0.75 mg or greater mRNA, 1 mg or greater mRNA, 1.25 mg or greater mRNA, 1.5 mg or greater mRNA, or 1.75 mg or greater mRNA.

27. The method of any one of claims 11-26, wherein the composition is formulated for a suppository of about 3 grams, about 2 grams, or about 1 gram.

28. The method of claim 27, wherein the composition is formulated for a suppository of about 3 grams.

29. The method of any one of claims 11-26, wherein the composition is formulated for a suppository having a volume of about 2.0 mL, about 3.5 mL, about 7.5 mL, or about 10.0 mL.

30. The method of any one of claims 11-29, wherein the suppository is refrigerated prior to administration.

31. The method of any one of the preceding claims, wherein the subject is first administered a permeability enhancer prior to the administering of the composition comprising mRNA.

32. The method of claim 31, wherein the permeability enhancer is administered to the subject about 30 minutes, about 1 hour, about 2.5 hours, about 5 hours, or about 12 hours prior to administering the composition comprising mRNA.

33. The method of claim 32, wherein the permeability enhancer is administered about 30 minutes prior to administering the composition comprising mRNA.

34. A method of delivery of messenger RNA (mRNA) to a subject for in vivo production of a protein or peptide in the subject, comprising administering to the subject by mucosal delivery a composition comprising an mRNA that encodes a protein or a peptide and is encapsulated within a lipid nanoparticle, and wherein the administering of the composition results in expression of the protein or peptide encoded by the mRNA that is detectable in the subject at least about 24 hours, about 48 hours, about 72 hours, or about 96 hours after administration.

35. The method of claim 34, wherein the mRNA is detectable in the subject's circulation, liver, kidney, colon, and/or rectum at least about 24 hours, about 48 hours, about 72 hours, or about 96 hours after administration.

36. The method of claim 34 or 35, wherein the mucosal delivery is rectal, vaginal, ocular, oral, or gastrointestinal.

37. The method of claim 36, wherein the oral delivery is buccal or sublingual.

38. The method of any one of claims 33-37, wherein the mucosal delivery is rectal.

39. The method of any one of claims 33-37, wherein the in vivo production of the protein or peptide is in the subject's circulation, liver, kidney, colon and/or rectum.

40. A suppository for rectal administration of mRNA, the suppository comprising:
a. mRNA encapsulated within a lipid nanoparticle, wherein the mRNA encodes a protein or peptide; and
b. gelatin.

41. The suppository of claim 40 comprising about 5% or more gelatin in water, 10% or more gelatin in water, 20% or more gelatin in water, 30% or more gelatin in water, or 50% or more gelatin in water.

42. The suppository of any one of claim 40 or 41, wherein the suppository does not comprise a lipid-based suppository component.

43. The suppository of claim 42, wherein the lipid-based suppository component is cocoa butter, theobroma oil, synthetic fats or synthetic bases.

44. The suppository of any one of claim 40-43, further comprising a permeability enhancer.

45. The suppository of claim 44, wherein the permeability enhancer is selected from bile salts, surfactants, fatty acids and derivatives, glycerides, chelators, salicylates, or polymers.

46. The suppository of any one of claims 40-45, wherein the fatty acids and derivatives are selected from sorbitan laurate, sodium caprate, sucrose, palitate, lauroyl choline, sodium myristate, or palmitoyl carnitine.

47. The suppository of any one of claims 40-45, wherein the permeability enhancer is a form of caprate.

48. The suppository of claim 47, wherein the caprate-based permeability enhancer is sodium caprate.

49. The suppository of 44, wherein the permeability enhancer is Labrasol®.

50. The suppository of any one of claims 40-49, further comprising glycerin and/or PEG.

51. The suppository of any one of claims 40-50, wherein the suppository softens or melts at about between 36 and 37° C.
